(19) United States
(12) Patent Application Publication
(10) Pub. No.: US 2011/0230551 A1
(43) Pub. Date: Sep. 22, 2011

(54) WITHAFERIN A ANALOGS AND USES THEREOF

(76) Inventors: Leslie Gunatilaka, Tucson, AZ (US); Ekanayake Mudiyanselage Kithsiri Wijeratne, Tucson, AZ (US); Ya-Ming Xu, Tucson, AZ (US); Luke Whitesell, Somerville, MA (US); Susan L. Lindquist, Chestnut Hill, MA (US)

(21) Appl. No.: 13/063,825

(22) PCT Filed: Sep. 15, 2009
(86) PCT No.: PCT/US09/05146

§ 371 Date: May 25, 2011

Related U.S. Application Data
(60) Provisional application No. 61/097,088, filed on Sep. 15, 2008.

(51) Int. Cl. A61K 31/351; C07D 407/08; C12N 5/00; A61P 25/28

(52) U.S. Cl. 514/460; 549/414; 435/325

(57) ABSTRACT

The present invention provides a novel class of withanolides that have been isolated from W. somnifera under aeroponic conditions or produced semi-synthetically from withanolide natural products. The invention also provides pharmaceutical compositions thereof and methods for using the same in proliferative diseases, neurodegenerative diseases, autoimmune, and inflammatory diseases.

### RELATED APPLICATIONS

The present application claims priority under 35 U.S.C. §119(e) to U.S. provisional patent application, U.S. Ser. No. 61/097,088, filed Sep. 15, 2008, which is incorporated herein by reference.

### BACKGROUND OF THE INVENTION

The roots of the medicinal plant Withania somnifera (L.) Dunal have been used for millennia in the Ayurvedic tradition of India for a variety of indications. The preparation of Withania roots is commonly known as ashwagandha. Ashwagandha has been shown to possess anti-inflammatory (Anbalagan, et al., Indian J. Exp. Biol. (1981) 19: 245-249), immunomodulatory (Ziauddin, et al., J. Ethnopharmacol. (1996) 50: 69-76; Dhuley, et al., J. Ethnopharmacol. (1997) 58: 15-20), cardioprotective (Dhuley, et al., J. Ethnopharmacol. (2000) 70: 57-63), antioxidant (Dhuley, et al., J. Ethnopharmacol. (1998) 60: 173-178), and anti-proliferative (Jayaprakasam, et al., Life Sci. (2003) 74: 125-132) activities (see also Mishra, et al., Ahern. Med. Rev. (2000) δ: 334-346; Gupta, et al., Pharmacog. Rev. (2007) 1: 129-136; Kumar, et al., Asian J. Chem. (2006) 18: 1401-1404).

The primary bioactive constituents of ashwagandha are known as withanolides. These compounds are structurally diverse steroidal compounds with an ergosterol skeleton in which C-22 and C-26 are oxidized to form a δ-lactone (Ray, et al., Prog. Chem. Org. Nat. Prod. (1994) 63: 1-106). Withaferin A, a withanolide, has been proposed to inhibit the actions of many targets, and its inhibitory activity may be cell-type specific. Possible biological targets of withaferin A and related withanolides are the actin bundling protein annexin II (Falsey, et al., Nat. Chem. Biol. (2006) 2: 33-38), the 20S proteasome (Yang, et al., Mol. Pharmacol. (2007) 71: 426-437), the intermediate filament protein vimentin (Bargagna-Mohan, et al., Chem. Biol. (2007) 14: 623-634), the transcription factor NFκB (Srinivasan, et al., Cancer Res. (2007) 67: 246-253), protein kinase C (Sen, et al., Cell Death Differ. (2007) 14: 358-367), and the Par-4-dependent apoptosis pathway (Kaileh, et al., J. Biol. Chem. (2007) 282: 4253-4264).

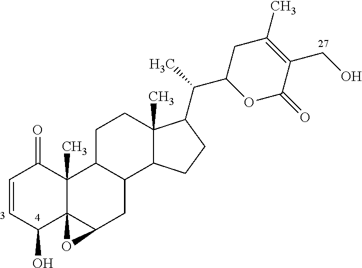

### Withaferin A

Since the withanolides have shown promising biological activities, there remains a need for identifying further related compounds with useful biological activities, especially those that are amenable to formulation.

### SUMMARY OF THE INVENTION

The present invention stems from the recognition that analogs of withaferin A may be useful in inducing the heat shock response and therefore may be useful in treating neurodegenerative disorders associated with protein aggregration. In addition, analogs of withaferin A may exhibit anti-proliferative/anti-survival properties useful in treating diseases such as cancer. The present invention provides a novel class of withanolides that have been isolated from W. somnifera or produced semi-synthetically from withanolide natural products. It also provides new methods for the aeroponic culture of W. somnifera to provide bulk quantities of biomass under conditions that allow improved yield and consistency of desired secondary metabolite production. Certain inventive compounds, such as certain compounds derived from aeroponically cultured biomass, have been found to activate the heat shock response in fibroblasts (FIG. 8) and have been found to inhibit cell proliferation/survival in MCF-7 breast cancer cells (FIG. 1). The inventive compounds also may be amenable to formulation for in vivo administration. For example, the inventive compounds may be more water soluble than known withanolides. Thus, the present invention represents an important advance in the field of withanolides.

In certain embodiments, inventive compounds are generally of the formula:

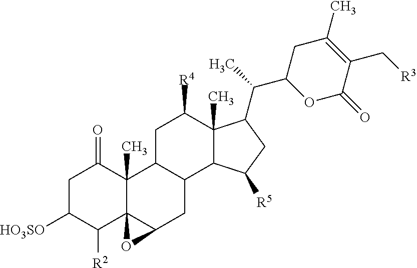

or a pharmaceutically acceptable salt thereof; wherein

R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R³, R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{C}; —SO₂R_{C}; or —C(R^{D})₃.

In certain embodiments, the inventive compound is of the formula:

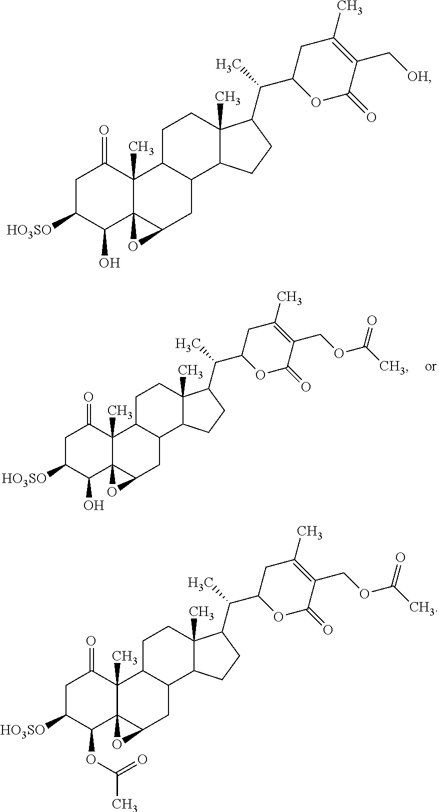

In certain embodiments, inventive compounds are generally of the formula:

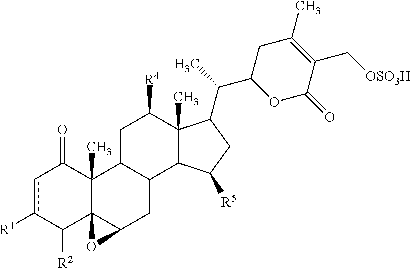

or a pharmaceutically acceptable salt thereof; wherein

[symbol] =denotes a single or double bond;

R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{D}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃;

R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

In certain embodiments, the inventive compound is of the formula:

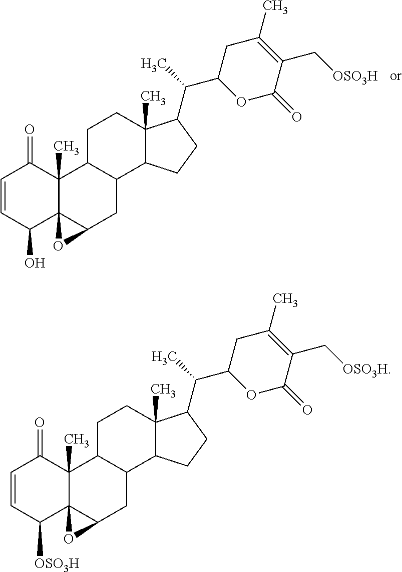

In certain embodiments, inventive compounds are generally of the formula:

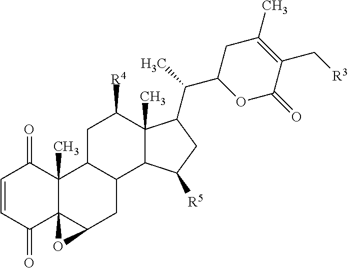

or a pharmaceutically acceptable salt thereof; wherein

R³, R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{C}; —SO₂R_{C}; or —C(R^{D})₃, wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

In certain embodiments, the inventive compound is of the formula:

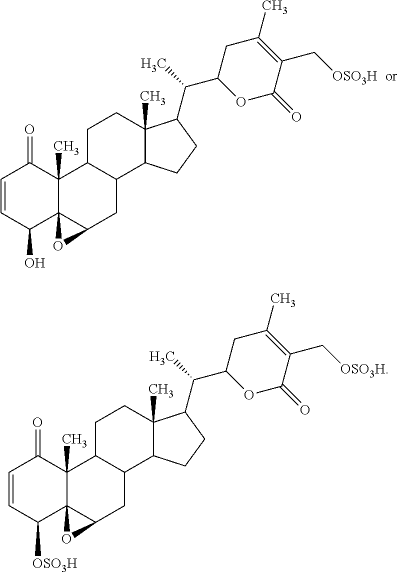

In certain embodiments, inventive compounds are generally of the formula:

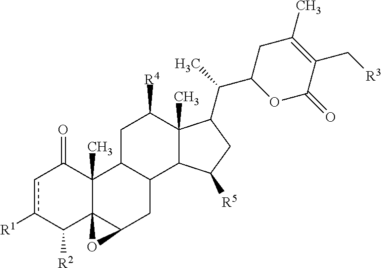

or a pharmaceutically acceptable salt thereof; wherein

[symbol] =denotes a single or double bond;

R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{D}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃;

R³, R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{C}; —SO₂R_{C}; or —C(R^{D})₃.

In certain embodiments, the inventive compound is of the formula:

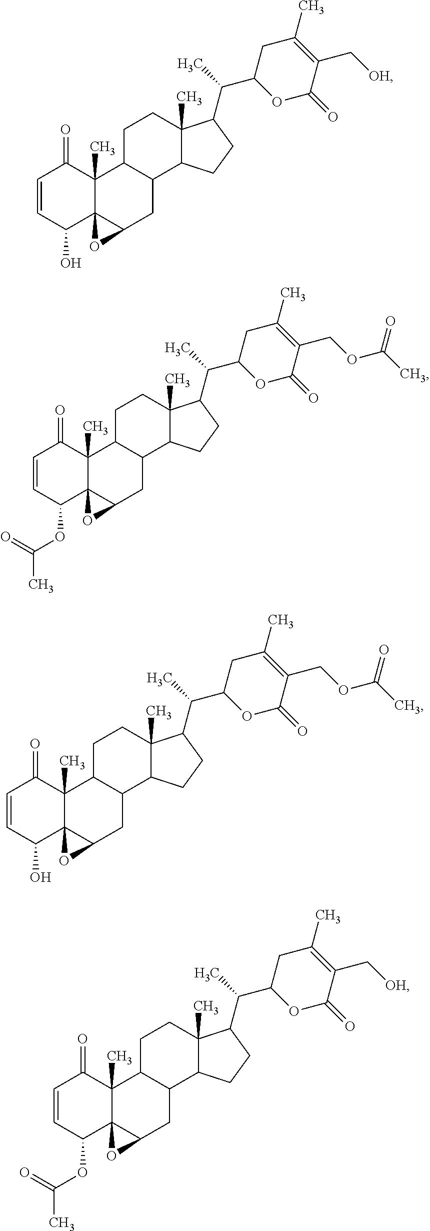

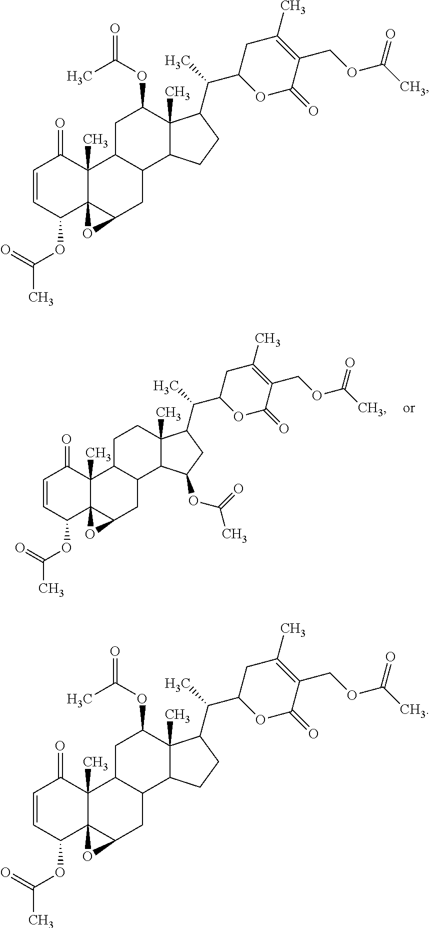

The compounds of the present invention may be isolated from W. somnifera. The compounds of the present invention may also be produced semi-synthetically from withanolide natural products (e.g., by modification of a hydroxyl group). The compounds of the present invention may also be produced by total synthesis.

In one aspect, the present invention provides pharmaceutical compositions comprising the inventive compounds. The pharmaceutical compositions may optionally include a pharmaceutically acceptable excipient. Any mode of administration including oral and parenteral administration of the inventive compound or pharmaceutical composition thereof may be used.

In another aspect, the present invention provides methods of treatment comprising the inventive compounds. The compounds of the invention or pharmaceutical compositions thereof may be used to treat any disease including proliferative diseases such as cancer and benign neoplasms, disorders involving neoangiogenesis, autoimmune diseases, inflammatory diseases, cardiovascular diseases, neurodegenerative diseases, and protein aggregation disorders. The compounds of the invention may be used to treat disease in humans and other animals including domesticated animals. The inventive compounds may also be used as probes of biological pathways. For example, the compounds of the invention may be used to inhibit proliferation of cells or induce the heat shock response in cells.

In another aspect, the present invention provides methods for isolating and synthesizing withanolides. In certain embodiments, withanolides are isolated from aeroponically grown biomass. In some embodiments, withanolides isolated from natural sources are further derivatized using synthetic methods including acetylation, oxidation, and reduction.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

The compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N.Y., 1962); Wilen, Tables of Resolving Agents and Optical Resolutions p. 268 (E.L. Eliel, Ed., Univ. of Notre Dame Press, Notre Dame, Ind. 1972).

It will be appreciated that the compounds of the present invention, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety. The present invention contemplates any and all such combinations in order to arrive at a stable substituent/moiety. Additional examples of generally applicable substitutents are illustrated by the specific embodiments shown in the Examples, which are described herein. For purposes of this invention, heteroatoms such as nitrogen may have hydrogen substituents and/or any suitable substituent as described herein which satisfy the valencies of the heteroatoms and results in the formation of a stable moiety.

As used herein, substituent names which end in the suffix “-ene” refer to a biradical derived from the removal of two hydrogen atoms from the substitutent. Thus, for example, acyl is acylene; alkyl is alkylene; alkeneyl is alkenylene; alkynyl is alkynylene; heteroalkyl is heteroalkylene, heteroalkenyl is heteroalkenylene, heteroalkynyl is heteroalkynylene, aryl is arylene, and heteroaryl is heteroarylene.

The term “acyl,” as used herein, refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “acyloxy” refers to a “substituted hydroxyl” of the formula (—ORⁱ), wherein Rⁱ is an optionally substituted acyl group, as defined herein, and the oxygen moiety is directly attached to the parent molecule.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substitutents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-15 carbon atoms. In another embodiment, the alkenyl group employed contains 2-10 carbon atoms. In still other embodiments, the alkenyl group contains 2-8 carbon atoms. In yet other embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “amino,” as used herein, refers to a group of the formula (—NH₂). A “substituted amino” refers either to a mono-substituted amine (—NHR^{h}) of a disubstitued amine (—NR^{h}₂), wherein the R^{h} substituent is any substitutent as described herein that results in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the di-substituted amino group (—NR^{h}₂) form a 5- to 6-membered hetereocyclic ring.

The term “alkoxy” refers to a “substituted hydroxyl” of the formula (—ORⁱ), wherein Rⁱ is an optionally substituted alkyl group, as defined herein, and the oxygen moiety is directly attached to the parent molecule.

The term “alkylthioxy” refers to a “substituted thiol” of the formula (—SR^{r}), wherein R^{r} is an optionally substituted alkyl group, as defined herein, and the sulfur moiety is directly attached to the parent molecule.

The term “alkylamino” refers to a “substituted amino” of the formula (—NR^{h}₂), wherein R^{h} is, independently, a hydrogen or an optionally substituted alkyl group, as defined herein, and the nitrogen moiety is directly attached to the parent molecule.

The term “aryl,” as used herein, refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “arylalkyl,” as used herein, refers to an aryl substituted alkyl group, wherein the terms “aryl” and “alkyl” are defined herein, and wherein the aryl group is attached to the alkyl group, which in turn is attached to the parent molecule. An exemplary arylalkyl group includes benzyl.

The term “aryloxy” refers to a “substituted hydroxyl” of the formula (—ORⁱ), wherein Rⁱ is an optionally substituted aryl group, as defined herein, and the oxygen moiety is directly attached to the parent molecule.

The term “arylamino,” refers to a “substituted amino” of the formula (—NR^{h}₂), wherein R^{h} is, independently, a hydrogen or an optionally substituted aryl group, as defined herein, and the nitrogen moiety is directly attached to the parent molecule.

The term “arylthioxy” refers to a “substituted thiol” of the formula (—SR^{r}), wherein R^{r} is an optionally substituted aryl group, as defined herein, and the sulfur moiety is directly attached to the parent molecule.

The term “azido,” as used herein, refers to a group of the formula (—N₃).

The term “cyano,” as used herein, refers to a group of the formula (—CN).

The terms “halo” and “halogen” as used herein refer to an atom selected from fluorine (fluoro, —F), chlorine (chloro, —Cl), bromine (bromo, —Br), and iodine (iodo, —I).

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl,” as used herein, refers to an alkyl moiety, as defined herein, which contain one or more oxygen, sulfur, nitrogen, phosphorus, or silicon atoms, e.g., in place of carbon atoms.

The term “heteroalkenyl,” as used herein, refers to an alkenyl moiety, as defined herein, which contain one or more oxygen, sulfur, nitrogen, phosphorus, or silicon atoms, e.g., in place of carbon atoms.

The term “heteroalkynyl,” as used herein, refers to an alkynyl moiety, as defined herein, which contain one or more oxygen, sulfur, nitrogen, phosphorus, or silicon atoms, e.g., in place of carbon atoms.

The term “heteroalkylamino” refers to a “substituted amino” of the formula (—NR^{h}₂), wherein R^{h} is, independently, a hydrogen or an optionally substituted heteroalkyl group, as defined herein, and the nitrogen moiety is directly attached to the parent molecule.

The term “heteroalkyloxy” refers to a “substituted hydroxyl” of the formula (—ORⁱ), wherein Rⁱ is an optionally substituted heteroalkyl group, as defined herein, and the oxygen moiety is directly attached to the parent molecule.

The term “heteroalkylthioxy” refers to a “substituted thiol” of the formula (—SR^{r}), wherein R^{r} is an optionally substituted heteroalkyl group, as defined herein, and the sulfur moiety is directly attached to the parent molecule.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroaryl,” as used herein, refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylene,” as used herein, refers to a biradical derived from an heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylamino” refers to a “substituted amino” of the (—NR^{h}₂), wherein R^{h} is, independently, a hydrogen or an optionally substituted heteroaryl group, as defined herein, and the nitrogen moiety is directly attached to the parent molecule.

The term “heteroaryloxy” refers to a “substituted hydroxyl” of the formula (—ORⁱ), wherein Rⁱ is an optionally substituted heteroaryl group, as defined herein, and the oxygen moiety is directly attached to the parent molecule.

The term “heteroarylthioxy” refers to a “substituted thiol” of the formula (—SR^{r}), wherein R^{r} is an optionally substituted heteroaryl group, as defined herein, and the sulfur moiety is directly attached to the parent molecule.

The term “hydroxy,” or “hydroxyl,” as used herein, refers to a group of the formula (—OH). A “substituted hydroxyl” refers to a group of the formula (—ORⁱ), wherein Rⁱ can be any substitutent which results in a stable moiety (e.g., a suitable hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “imino,” as used herein, refers to a group of the formula (═NR^{r}), wherein R^{r} corresponds to hydrogen or any substitutent as described herein, that results in the formation of a stable moiety (for example, a suitable amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted). In certain embodiments, imino refers to ═NH wherein R^{r} is hydrogen.

The term “isocyano,” as used herein, refers to a group of the formula (—NC).

The term “nitro,” as used herein, refers to a group of the formula (—NO₂).

The term “oxo,” as used herein, refers to a group of the formula (═O).

The term “stable moiety,” as used herein, preferably refers to a moiety which possess stability sufficient to allow manufacture, and which maintains its integrity for a sufficient period of time to be useful for the purposes detailed herein.

A “suitable amino-protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylypethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl) 6 chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridypethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenypamine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “suitable carboxylic acid protecting group,” or “protected carboxylic acid,” as used herein, are well known in the art and include those described in detail in Greene (1999). Examples of suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

A “suitable hydroxyl protecting group” as used herein, is well known in the art and include those described in detail in Greene (1999). Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-tri chloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “suitable thiol protecting group,” as used herein, are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of suitable ester groups include formates, acetates, proprionates, pentanoates, crotonates, and benzoates. Specific examples of suitable ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl groups include trimethylsilyl, triethylsilyl, t-butyldimethylsilyl, t-butyldiphenylsilyl, triisopropylsilyl ether, and other trialkylsilyl ethers. Examples of suitable alkyl groups include methyl, benzyl, p-methoxybenzyl, 3,4-dimethoxybenzyl, trityl, t-butyl, and allyl ether, or derivatives thereof. Examples of suitable arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The term “thio,” or “thiol,” as used herein, refers to a group of the formula (—SH). A “substituted thiol” refers to a group of the formula (—SR^{r}), wherein W can be any substituent that results in the formation of a stable moiety (e.g., a suitable thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “thiooxo,” as used herein, refers to a group of the formula (═S).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The following definitions are more general terms used throughout the present application:

The term “subject,” as used herein, refers to any animal. In certain embodiments, the subject is a mammal. In certain embodiments, the term “subject”, as used herein, refers to a human (e.g., a man, a woman, or a child).

The terms “administer,” “administering,” or “administration,” as used herein refers to implanting, absorbing, ingesting, injecting, or inhaling the inventive compound.

The terms “treat” or “treating,” as used herein, refers to partially or completely alleviating, inhibiting, ameliorating, and/or relieving the disease or condition from which the subject is suffering.

The terms “effective amount” and “therapeutically effective amount,” as used herein, refer to the amount or concentration of an inventive compound, that, when administered to a subject, is effective to at least partially treat a condition from which the subject is suffering (e.g., a neurodegenerative disease).

As used herein, the term “withanolide” refers to a natural product or analog thereof isolated from Withania somnifera or another Withania species.

### BRIEF DESCRIPTION OF THE DRAWINGS

FIG. 1. Concentration- and time-dependent cell proliferation/survival inhibition by 2,3-dihydrowithaferin A-3β-O-sulfate (WA-SO4) and withaferin A (WA). MCF-7 breast cancer cells were exposed to the indicated concentrations of compounds and relative viable cell number determined by dye reduction assay after the time intervals noted. Data are representative of three independent experiments.

FIG. 2. Conversion of 2,3-dihydrowithaferin A-3β-O-sulfate (solid circles) to withaferin A (solid squares) in cell culture medium. (A) Incubation of 2,3-dihydrowithaferin A-3β-O-sulfate in DMEM supplemented with 10% fetal bovine serum. (B) Incubation in cysteine/methionine-free DMEM supplemented with 10% fetal bovine serum. The concentration of 2,3-dihydrowithaferin A-3β-O-sulfate (solid circles) and its conversion to withaferin A (solid squares) was measured over time by HPLC using an external standard curve method.

FIGS. 3A-3D. Induction of F-actin aggregation. Following incubation with test compounds for the indicated intervals, WI-38 fibroblasts were fixed and stained with fluorescently-labeled phalloidin (green signal) to visualize the actin cytoskeleton. Cells were counterstained with DAPI (blue signal) to identify their nuclei. All images were acquired using the same magnification and exposure conditions. Data are representative of two independent experiments. (A) DMSO, 24 h incubation. (B) Withaferin A, 4 h. (C) 2,3-Dihydrowithaferin A-3β-O-sulfate, 4 h. (D) 2,3-Dihydrowithaferin A-3β-O-sulfate, 24 h.

FIG. 4. Inhibition of tumor cell invasion/migration. PC-3M (prostate) and CHP-100 (Ewing's sarcoma) tumor cells were seeded into Matrigel-coated invasion chambers and exposed to increasing concentrations of withaferin A (WA) or DMSO for 24 hours. Relative viable cell number and invasion were determined by MTT assay. Results are presented as % compared to wells exposed to DMSO alone. Results shown are representative of three independent experiments.

FIG. 5. Concentration-dependent inhibition of tumor cell growth in vitro. Ewing's sarcoma cells (CHP-100) were exposed to increasing concentrations of withaferin A (WA) or the indicated semi-synthetic derivatives for 72 hours. Relative viable cell number was quantified by MTT assay. Results are presented as a percentage compared to wells exposed to DMSO alone. Circles: withaferin A; squares: epi-withaferin A; triangles: 4,27-di-O-acetyl withaferin A; X: 4,27-di-O-acetyl epi-withaferin A. Points: mean of triplicate determinations; Error bars: s.d.

FIG. 6. Inhibition of endothelial cell network formation in cell culture. Human umbilical vein endothelial cells (HUVEC) were seeded into Matrigel-coated wells and allowed to adhere for 1 h. Adherent cells were exposed to the indicated concentrations of withaferin A, epi-withaferin A (α-WA) or an equal volume of solvent vehicle (DMSO) for 16 h. Cells were washed 1× in PBS and fixed to visualize tube formation by light microscopy.

FIG. 7. Withaferin A inhibits tumor vascularization. SCID mice bearing CHP-100 tumor xenografts were treated with IP injections of DMSO (50 μl for 10 days) or withaferin A (WA) (7.5 mg/kg for 2 days and 3.5 mg/kg for 8 days) after tumor establishment. Upper panels: Microvessel density (MVD) of excised tumors from each treatment group (DMSO, left panel; WA, right panel) was visualized using formalin-fixed, paraffin-embedded material sectioned at four to five microns-thick. Samples were stained with antibody to CD-34 and detected through the use of indirect avidin-biotin-peroxidase methodology. Nuclei were counter-stained with hematoxylin and sections were evaluated by light microscopy. Lower Panel: Vascular density was quantitated by light microscopy based on the methods of the Weidner research group (**P value <0.01). Unpaired Student's t-test used to calculate P values. Error bars: s.d.

FIG. 8. Heat shock reporter induction measured by micro plate fluorimeter. Reporter cells stably transduced with a plasmid encoding enhanced green fluorescent protein (EGFP) under the control of a minimal heat shock response element were exposed to 2,3-dihydrowithaferin A-3β-O-sulfate (WA-SO4) or withaferin A (WA) at the indicated concentrations overnight. Relative fluorescence units (RFU) per well were determined as a measure of reporter activation. Each point represents the mean of nine determinations from three independent experiments.

FIG. 9. HSF1-dependent induction of the heat shock response. Immortalized mouse embryo fibroblasts derived from mice in which Hsf1 was knocked out [Hsf1 (−)] or their wild type littermates; [Hsf1(+)] were exposed overnight to DMSO (0.2%) (lanes 1 and 4), geldanamycin (GA, 0.5 μM) (lanes 2 and 5), or withaferin A (WA) (2 μM) (lanes 3 and 6). Equal amounts of total cellular protein were immunoblotted for relative levels of a highly inducible heat shock protein (Hsp72).

FIG. 10. Heat-shock protein induction following Withaferin A (WA) administration to mice. WA was formulated in DMSO/Cremophor/Saline vehicle and injected intraperitoneally (IP) at a total dose of 18 mg/kg/day, either as a single injection (WA X1) or divided in two 9 mg/kg injections (WA X2) spaced 6 hrs apart. Control animals received an equal volume of vehicle alone as a single injection. Mice were sacrificed 16 hours after the last injection, organs harvested and protein lysates prepared for immunoblotting. Equal loading of samples was confirmed by staining membranes for total protein (left panels). The relative tissue levels of Hsp72, Hsp27 and Annexin 2 (a putative target of Withaferin A) were determined by probing with specific antibodies as indicated. Note the robust induction of heat shock protein levels in spleen (Annexin 2 positive), but not normal brain (Annexin 2 negative) associated with prior Withaferin A exposure.

FIG. 11. Stable and specific binding of Annexin 2 by withaferin A. Biotinylated withaferin A (WA) was captured on NeutrAvidin (NA)-coated beads and incubated with pre-cleared whole cell extract that had been previously supplemented with WA (40 μM) or an equal volume of DMSO. After low stringency washes, proteins bound to beads were eluted, size fractioned by SDS-PAGE, stained with Sypro Ruby, and visualized by UV illumination. The arrowhead indicates the position of a 36 kDa band effectively competed away by WA. Pre-clear lane: Proteins in whole cell extract that bound non-specifically to NA beads alone (no immobilized WA present) during pre-clearing incubation.

FIG. 12. Neuronal survival and increase in neurite length as assessed by calcein-A staining which is taken up by viable neurons. A. In the absence of BDNF (-BDNF) there is approximately 40% reduction in viable motor neurons compared to wells with BDNF present (+BDNF). Addition of withaferin A (WA) to the motor neuron cultures plated in the absence of BDNF results in a 50% reduction in cell death at 200 nM (0.2 μM) concentration of WA and a 75% reduction in cell death at 400 nM (0.4 μM) as assessed after 24 hours. These data were acquired using Metamorph® software. B. Graph showing percent protection. C. Motor neuron viability assay of WA. D. WA increases neurite length (normalized to control) measured at the end of the experiment.

FIG. 13. Schematic diagram of glutathione depletion co-culture model for astrocyte-specific Nrf2-mediated neuronal protection from oxidative stress.

FIG. 14. A. Withaferin A (WA) mediates astrocyte-dependent neuronal protection. Primary astrocytes cultured from the cerebral cortices of postnatal rat pups, were treated with WA for 24 h, and were washed with serum-containing media to completely remove WA from the culture media. Primary neurons derived from E17 rat fetuses were then plated directly on the astrocyte monolayer in the presence or absence of 4 mM homocysteic acid (HCA) to induce oxidative stress-mediated neuronal death. 48 h later neuronal viability was assessed by quantifying the neuronal marker MAP-2. B. WA-mediated neuroprotection from oxidative stress.

FIG. 15. Protection of PC12 cells from toxicity caused by the inducible expression of expanded exon1 polyQ (103 glutamines) fused to the marker EGFP (HttQ103). Cells were exposed to withaferin A (WA) in the presence (induced +) or absence (induced −) of the ecdysone analog tebufenozide (1 uM). After 48 hour incubation, MTT assay was performed to assess metabolic activity as an indicator of relative viable cell number. There was a statistically significant difference between the DMSO+tebufenozide and the DMSO−tebufenozide, while no statistically significant difference was found between induced and uninduced cultures in the presence of WA treatment, demonstrating the ability of WA to protect PC12 cells from toxicity in this model.

### DETAILED DESCRIPTION OF CERTAIN EMBODIMENTS OF THE INVENTION

The present invention provides novel withanolides. Such compounds may be isolated from W. somnifera or produced semi-synthetically from natural products of W. somnifera (e.g., withaferin A). In certain embodiments, the inventive compound is isolated from aeroponically grown W. somnifera. The inventive compounds typically include a steroid core with an ergosterol skeleton as shown herein. The compounds of the present invention are useful in the treatment of proliferative diseases such as cancer, benign neoplasms, and diseases involving neoangiogenesis. The compounds of the present invention are also useful in the treatment of protein aggregation disorders. The present invention also provides pharmaceutical compositions and methods of using the inventive compounds for the treatment of various diseases (e.g., neurodegenerative diseases).

### Compounds

Compounds of the present invention include withanolides and analogs thereof. Particularly useful compounds of the present invention include those with biological activity. The inventive compounds have been found to have a variety of biological activities. In certain embodiments, the compounds of the invention have anti-proliferative activity. In certain embodiments, the compounds of the invention have cytotoxic activity. In certain embodiments, the compounds of the invention modulate the heat shock response. In certain embodiments, the compounds modulate annexin II. In certain embodiments, the compounds inhibit vimentin. In certain embodiments, the compounds inhibit NFκB activation. In certain embodiments, the compounds inhibit protein kinase C. In certain embodiments, the compounds induce apoptosis. In certain embodiments, the compound have an IC₅₀ of less than approximately 10 μM, e.g., less than approximately 1 μM, e.g., less than approximately 0.1 μM, or e.g., less than approximately 0.01 μM. The inventive compounds may be useful in the treatment of a variety of diseases. In certain embodiments, the compounds are useful in the treatment of proliferative diseases such as cancer and other neoplasms. Certain compounds are also useful in treating inflammatory diseases or autoimmune diseases. In certain embodiments, the compounds are useful in the treatment of cardiovascular diseases, diseases involving angiogenesis, neurodegenerative diseases, or protein aggregation disorders. Certain compounds of the invention are also useful as radiosensitizers. In certain embodiments, an inventive compound has greater solubility in water and other aqueous media than does withaferin A.

In certain embodiments, the invention provides a compound of formula (I) or a pharmaceutically acceptable salt thereof:

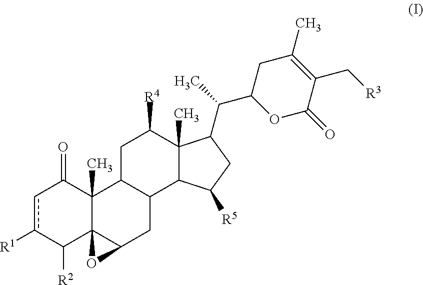

wherein

[symbol] denotes a single or double bond;

R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{D}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R² is ═O or —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; and

R³, R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

In certain embodiments, [symbol] is a double bond. In certain embodiments, [symbol] is a single bond.

In certain embodiments, R¹ of formula I is hydrogen. In certain other embodiments, R¹ of formula I is hydroxyl. In certain embodiments, R¹ of formula I is alkoxy. In certain embodiments, R¹ of formula I is a protected hydroxyl group. In certain embodiments, R¹ of formula I is phosphate. In certain embodiments, R¹ of formula I is sulfate. In certain other embodiments, R¹ of formula I is acetate.

In certain embodiments, R² of formula I is hydrogen. In certain other embodiments, R² of formula I is hydroxyl. In certain embodiments, R² of formula I is alkoxy. In certain embodiments, R² of formula I is a protected hydroxyl group. In certain embodiments, R² of formula I is phosphate. In certain embodiments, R² of formula I is sulfate. In certain other embodiments, R² of formula I is acetate.

In certain embodiments, R³ of formula I is hydrogen. In certain other embodiments, R³ of formula I is hydroxyl. In certain embodiments, R³ of formula I is alkoxy. In certain embodiments, R³ of formula I is a protected hydroxyl group. In certain embodiments, R³ of formula I is phosphate. In certain embodiments, R³ of formula I is sulfate. In certain other embodiments, R³ of formula I is acetate.

In certain embodiments, R⁴ of formula I is hydrogen. In certain other embodiments, R⁴ of formula I is hydroxyl. In certain embodiments, R⁴ of formula I is alkoxy. In certain embodiments, R⁴ of formula I is a protected hydroxyl group. In certain embodiments, R⁴ of formula I is phosphate. In certain embodiments, R⁴ of formula I is sulfate. In certain other embodiments, R⁴ of formula I is acetate.

In certain embodiments, R⁵ of formula I is hydrogen. In certain other embodiments, R⁵ of formula I is hydroxyl. In certain embodiments, R⁵ of formula I is alkoxy. In certain embodiments, R⁵ of formula I is a protected hydroxyl group. In certain embodiments, R⁵ of formula I is phosphate. In certain embodiments, R⁵ of formula I is sulfate. In certain other embodiments, R⁵ of formula I is acetate.

In certain embodiments, R⁴ and R⁵ of formula I are both hydrogen. In certain embodiments, only one of R⁴ and R⁵ are hydrogen. In certain embodiments, at least one of R⁴ and R⁵ is hydrogen.

In certain embodiments, compounds of the invention are of the formula:

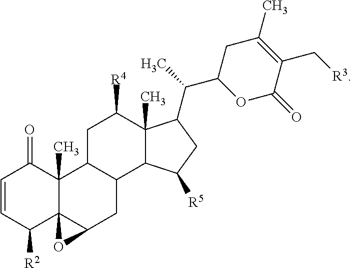

In certain embodiments, compounds of the invention are of the formula:

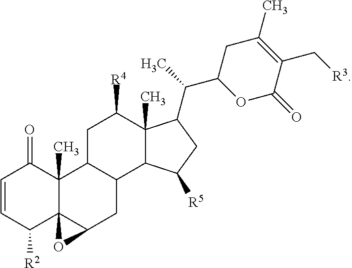

In certain embodiments, compounds of the invention are of the formula:

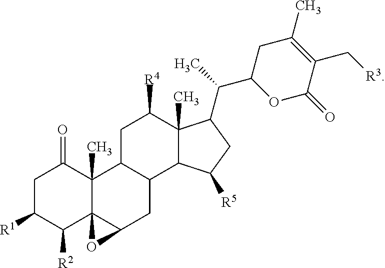

In certain embodiments, compounds of the invention are of the formula:

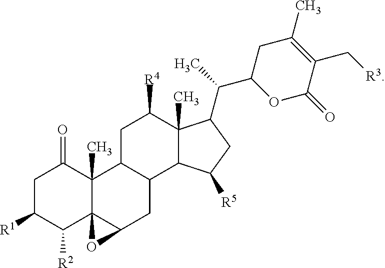

In certain embodiments, compounds of the invention are of the formula:

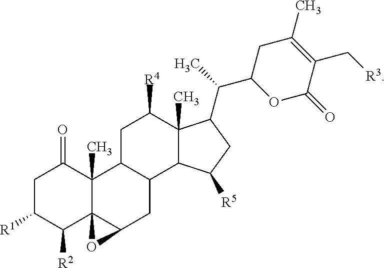

In certain embodiments, compounds of the invention are of the formula:

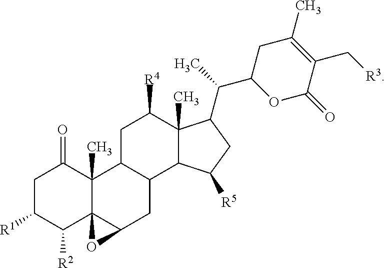

In certain embodiments, compounds of the invention are of the formula:

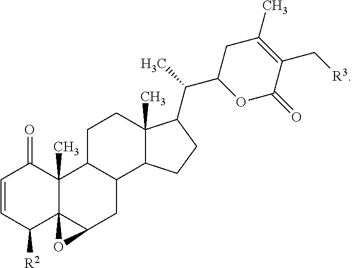

In certain embodiments, compounds of the invention are of the formula:

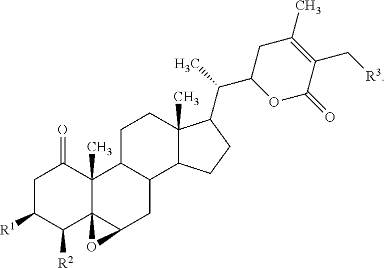

In certain embodiments, the invention provides a compound of formula (II) or a pharmaceutically acceptable salt thereof:

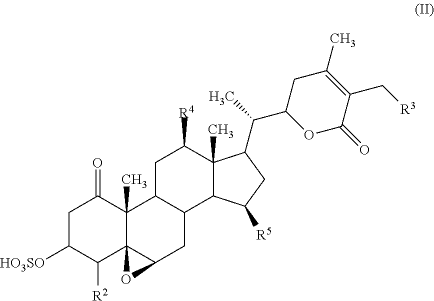

wherein

R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R³, R⁴ and R⁵ are each independently hydrogen or —OR^{S}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

In certain embodiments, R² of formula II is hydrogen. In certain other embodiments, R² of formula II is hydroxyl. In certain embodiments, R² of formula II is alkoxy. In certain embodiments, R² of formula II is a protected hydroxyl group. In certain embodiments, R² of formula II is phosphate. In certain embodiments, R² of formula II is sulfate. In certain other embodiments, R² of formula II is acetate.

In certain embodiments, R³ of formula II is hydrogen. In certain other embodiments, R³ of formula II is hydroxyl. In certain embodiments, R³ of formula II is alkoxy. In certain embodiments, R³ of formula II is a protected hydroxyl group. In certain embodiments, R³ of formula II is phosphate. In certain embodiments, R³ of formula II is sulfate. In certain other embodiments, R³ of formula II is acetate.

In certain embodiments, R⁴ of formula II is hydrogen. In certain other embodiments, R⁴ of formula II is hydroxyl. In certain embodiments, R⁴ of formula II is alkoxy. In certain embodiments, R⁴ of formula II is a protected hydroxyl group. In certain embodiments, R⁴ of formula II is phosphate. In certain embodiments, R⁴ of formula I is sulfate. In certain other embodiments, R⁴ of formula II is acetate.

In certain embodiments, R⁵ of formula II is hydrogen. In certain other embodiments, R⁵ of formula II is hydroxyl. In certain embodiments, R⁵ of formula II is alkoxy. In certain embodiments, R⁵ of formula II is a protected hydroxyl group. In certain embodiments, R⁵ of formula II is phosphate. In certain embodiments, R⁵ of formula II is sulfate. In certain other embodiments, R⁵ of formula II is acetate.

In certain embodiments, R⁴ and R⁵ of formula II are both hydrogen. In certain embodiments, only one of R⁴ and R⁵ are hydrogen. In certain embodiments, at least one of R⁴ and R⁵ is hydrogen.

In certain embodiments, compounds of the invention are of the formula:

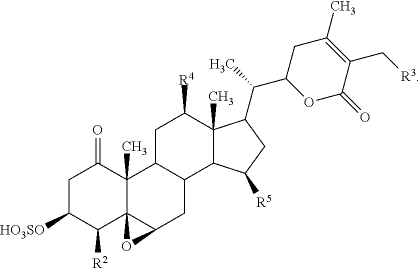

In certain embodiments, compounds of the invention are of the formula:

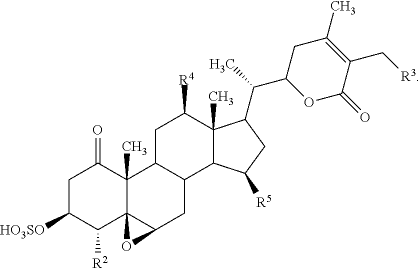

In certain embodiments, compounds of the invention are of the formula:

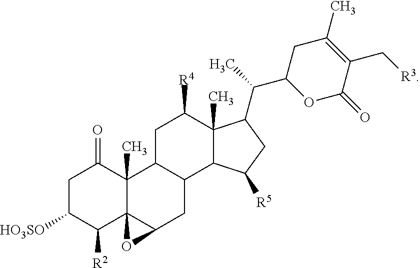

In certain embodiments, compounds of the invention are of the formula:

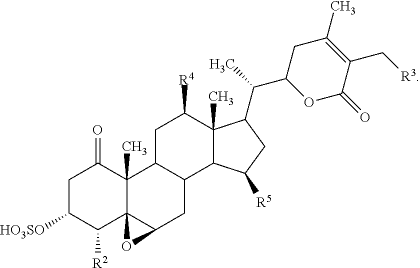

In certain embodiments, compounds of the invention are of the formula:

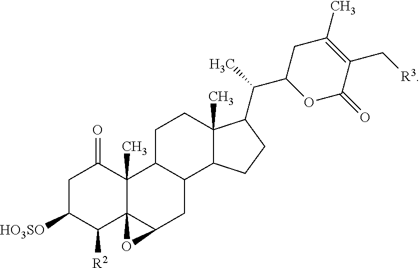

In certain embodiments, R² and R³ are —OR^{B}, where R^{B} is hydrogen or acetyl.

Exemplary compounds of the invention include:

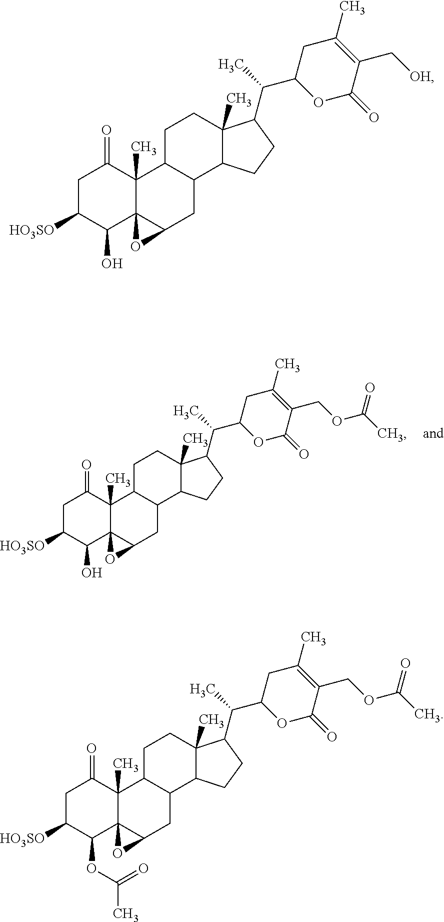

In one embodiment, the inventive compound is of the formula:

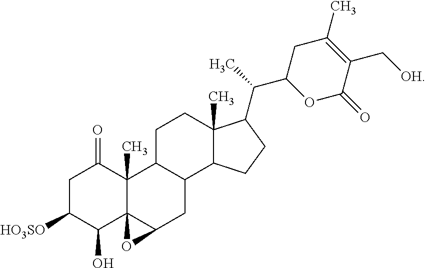

In certain embodiments, the invention provides a compound of formula (III) or a pharmaceutically acceptable salt thereof:

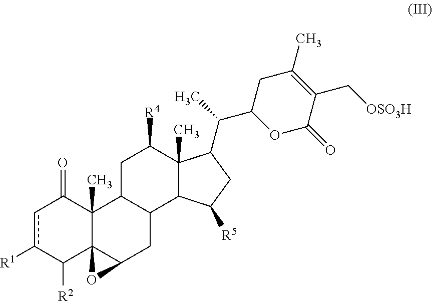

wherein

[symbol] denotes a single or double bond;

R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{D}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R² is ═O or —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; and

R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

In certain embodiments, [symbol] is a double bond. In certain embodiments, [symbol] is a single bond.

In certain embodiments, R¹ of formula III is hydrogen. In certain other embodiments, R¹ of formula III is hydroxyl. In certain embodiments, R¹ of formula III is alkoxy. In certain embodiments, R¹ of formula III is a protected hydroxyl group. In certain embodiments, R¹ of formula III is phosphate. In certain embodiments, R¹ of formula I is sulfate. In certain other embodiments, R¹ of formula III is acetate.

In certain embodiments, R² of formula III is hydrogen. In certain other embodiments, R² of formula III is hydroxyl. In certain embodiments, R² of formula III is alkoxy. In certain embodiments, R² of formula III is a protected hydroxyl group. In certain embodiments, R² of formula III is phosphate. In certain embodiments, R² of formula III is sulfate. In certain other embodiments, R² of formula III is acetate.

In certain embodiments, R⁴ of formula III is hydrogen. In certain other embodiments, R⁴ of formula III is hydroxyl. In certain embodiments, R⁴ of formula III is alkoxy. In certain embodiments, R⁴ of formula III is a protected hydroxyl group. In certain embodiments, R⁴ of formula III is phosphate. In certain embodiments, R⁴ of formula III is sulfate. In certain other embodiments, R⁴ of formula III is acetate.

In certain embodiments, R⁵ of formula III is hydrogen. In certain other embodiments, R⁵ of formula III is hydroxyl. In certain embodiments, R⁵ of formula III is alkoxy. In certain embodiments, R⁵ of formula III is a protected hydroxyl group. In certain embodiments, R⁵ of formula III is phosphate. In certain embodiments, R⁵ of formula III is sulfate. In certain other embodiments, R⁵ of formula III is acetate.

In certain embodiments, R⁴ and R⁵ of formula III are both hydrogen. In certain embodiments, only one of R⁴ and R⁵ are hydrogen. In certain embodiments, at least one of R⁴ and R⁵ is hydrogen.

In certain embodiments, compounds of the invention are of the formula:

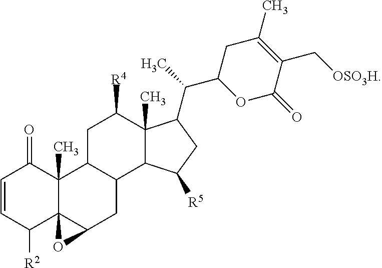

In certain embodiments, R² is —OAc. In certain embodiments, R² and R⁴ are —OR^{B}, and R⁵ is hydrogen. In certain embodiments, R² and R⁴ are —OH. In certain embodiments, R² and R⁴ are —OAc. In certain embodiments, R² and R⁵ are —OR^{B}, and R⁴ is hydrogen. In certain embodiments, R² and R⁵ are —OH. In certain embodiments, R² and R⁵ are —OAc.

In certain embodiments, compounds of the invention are of the formula:

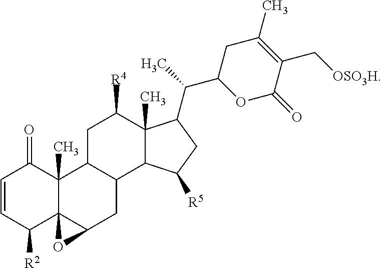

In certain embodiments, R² is —OAc. In certain embodiments, R² and R⁴ are —OR^{B}, and R⁵ is hydrogen. In certain embodiments, R² and R⁴ are —OH. In certain embodiments, R² and R⁴ are —OAc. In certain embodiments, R² and R⁵ are —OR^{B}, and R⁴ is hydrogen. In certain embodiments, R² and R⁵ are —OH. In certain embodiments, R² and R⁵ are —OAc.

In certain embodiments, compounds of the invention are of the formula:

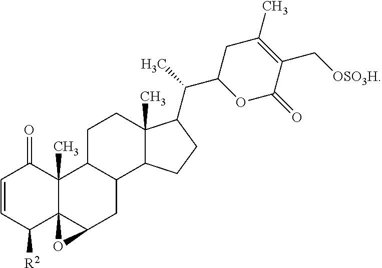

In certain embodiments, R² is —OAc. In certain embodiments, R² and R⁴ are —OR^{B}, and R⁵ is hydrogen. In certain embodiments, R² and R⁴ are —OH. In certain embodiments, R² and R⁴ are —OAc. In certain embodiments, R² and R⁵ are —OR^{B}, and R⁴ is hydrogen. In certain embodiments, R² and R⁵ are —OH. In certain embodiments, R² and R⁵ are —OAc.

In certain embodiments, compounds of the invention are of the formula:

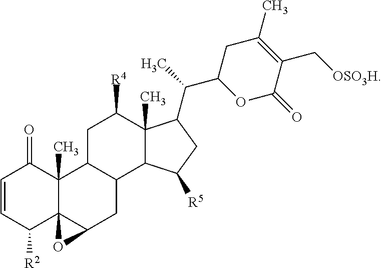

In certain embodiments, R² is —OAc. In certain embodiments, R² and R⁴ are —OR^{B}, and R⁵ is hydrogen. In certain embodiments, R² and R⁴ are —OH. In certain embodiments, R² and R⁴ are —OAc. In certain embodiments, R² and R⁵ are —OR^{B}, and R⁴ is hydrogen. In certain embodiments, R² and R⁵ are —OH. In certain embodiments, R² and R⁵ are —OAc.

In certain embodiments, compounds of the invention are of the formula:

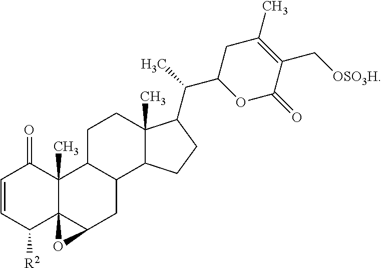

In certain embodiments, R² is —OR^{B}. In certain embodiments, R² is —OH. In certain embodiments, R² is —OAc.

In certain embodiments, compounds of the invention are of the formula:

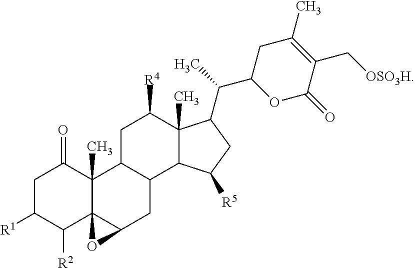

In some embodiments, R¹ is —OSO₃H. In some embodiments, R² and R³ are independently —OH or —OAc.

In certain embodiments, compounds of the invention are of the formula:

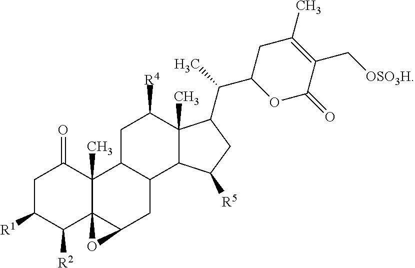

In some embodiments, R¹ is —OSO₃H. In some embodiments, R² and R³ are independently —OH or —OAc.

In certain embodiments, compounds of the invention are of the formula:

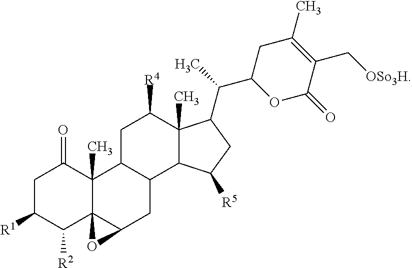

In some embodiments, R¹ is —OSO₃H. In some embodiments, R² and R³ are independently —OH or —OAc.

In certain embodiments, compounds of the invention are of the formula:

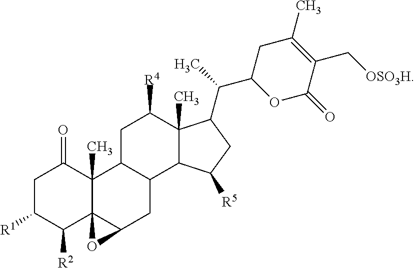

In some embodiments, R¹ is —OSO₃H. In some embodiments, R² and R³ are independently —OH or —OAc.

In certain embodiments, compounds of the invention are of the formula:

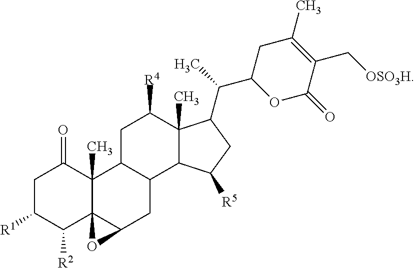

In some embodiments, R¹ is —OSO₃H. In some embodiments, R² and R³ are independently —OH or —OAc.

In certain embodiments, compounds of the invention are of the formula:

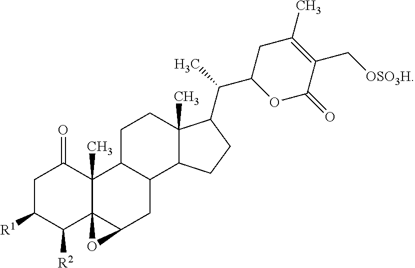

In some embodiments, R¹ is —OSO₃H. In some embodiments, R² and R³ are independently —OH or —OAc.

Exemplary compounds of the invention include:

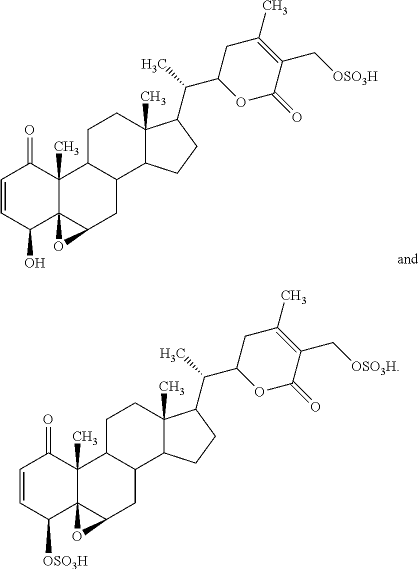

In certain embodiments, the invention provides a compound of formula (IV) or a pharmaceutically acceptable salt thereof:

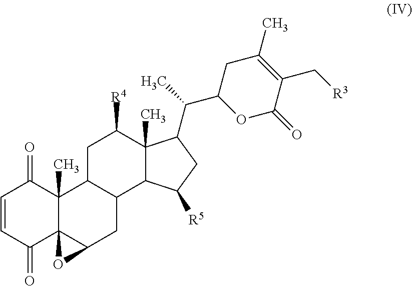

wherein

R³, R⁴ and R⁵ are each independently hydrogen or —OR^{C}, where each occurrence of R^{C} is independently hydrogen, —SO₃H; —PO₃H₂; —C(═O)R^{D}; —C(═O)N(R^{D})₂; —CO₂R^{D}; —SOR_{C}; —SO₂R_{C}; or —C(R^{D})₃.

In certain embodiments, R³ of formula IV is hydrogen. In certain other embodiments, R³ of formula I is hydroxyl. In certain embodiments, R³ of formula IV is alkoxy. In certain embodiments, R³ of formula IV is a protected hydroxyl group. In certain embodiments, R³ of formula IV is phosphate. In certain embodiments, R³ of formula IV is sulfate. In certain other embodiments, R³ of formula IV is acetate.

In certain embodiments, R⁴ of formula IV is hydrogen. In certain other embodiments, R⁴ of formula IV is hydroxyl. In certain embodiments, R⁴ of formula IV is alkoxy. In certain embodiments, R⁴ of formula IV is a protected hydroxyl group. In certain embodiments, R⁴ of formula IV is phosphate. In certain embodiments, R⁴ of formula IV is sulfate. In certain other embodiments, R⁴ of formula IV is acetate.

In certain embodiments, R⁵ of formula IV is hydrogen. In certain other embodiments, R⁵ of formula IV is hydroxyl. In certain embodiments, R⁵ of formula IV is alkoxy. In certain embodiments, R⁵ of formula IV is a protected hydroxyl group. In certain embodiments, R⁵ of formula IV is phosphate. In certain embodiments, R⁵ of formula IV is sulfate. In certain other embodiments, R⁵ of formula IV is acetate.

In certain embodiments, R⁴ and R⁵ of formula IV are both hydrogen. In certain embodiments, only one of R⁴ and R⁵ are hydrogen. In certain embodiments, at least one of R⁴ and R⁵ is hydrogen.

Exemplary compounds of the invention include:

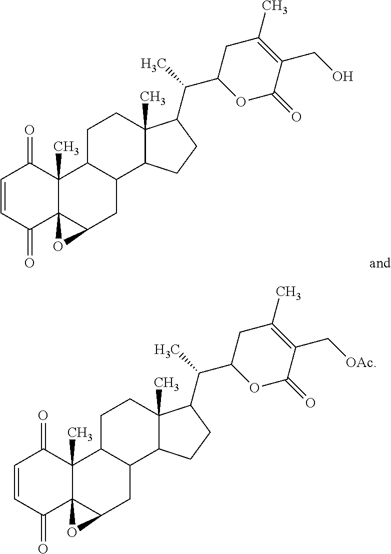

In certain embodiments, the invention provides a compound of formula (V) or a pharmaceutically acceptable salt thereof:

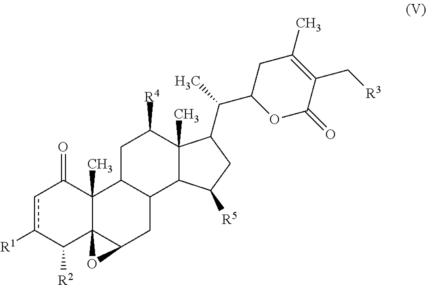

wherein

[symbol] denotes a single or double bond;

R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{D}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R² is ═O or —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; and

R³, R⁴ and R⁵ are each independently hydrogen or —OR^{S}, where each occurrence of R^{C} is independently hydrogen, —SO₃H; —PO₃H₂; —C(═O)R^{D}; —C(═O)N(R^{D})₂; —CO₂R^{D}; —SOR_{C}; —SO₂R_{C}; or —C(R^{D})₃.

In certain embodiments, [symbol] is a double bond. In certain embodiments, [symbol] is a single bond.

In certain embodiments, R¹ of formula V is hydrogen. In certain other embodiments, R¹ of formula V is hydroxyl. In certain embodiments, R¹ of formula V is alkoxy. In certain embodiments, R¹ of formula V is a protected hydroxyl group. In certain embodiments, R¹ of formula V is phosphate. In certain embodiments, R¹ of formula V is sulfate. In certain other embodiments, R¹ of formula V is acetate.

In certain embodiments, R² of formula V is hydrogen. In certain other embodiments, R² of formula V is hydroxyl. In certain embodiments, R² of formula V is alkoxy. In certain embodiments, R² of formula V is a protected hydroxyl group. In certain embodiments, R² of formula V is phosphate. In certain embodiments, R² of formula V is sulfate. In certain other embodiments, R² of formula V is acetate.

In certain embodiments, R³ of formula V is hydrogen. In certain other embodiments, R³ of formula V is hydroxyl. In certain embodiments, R³ of formula V is alkoxy. In certain embodiments, R³ of formula V is a protected hydroxyl group. In certain embodiments, R³ of formula V is phosphate. In certain embodiments, R³ of formula V is sulfate. In certain other embodiments, R³ of formula V is acetate.

In certain embodiments, R⁴ of formula V is hydrogen. In certain other embodiments, R⁴ of formula V is hydroxyl. In certain embodiments, R⁴ of formula V is alkoxy. In certain embodiments, R⁴ of formula V is a protected hydroxyl group. In certain embodiments, R⁴ of formula V is phosphate. In certain embodiments, R⁴ of formula V is sulfate. In certain other embodiments, R⁴ of formula V is acetate.

In certain embodiments, R⁵ of formula V is hydrogen. In certain other embodiments, R⁵ of formula V is hydroxyl. In certain embodiments, R⁵ of formula V is alkoxy. In certain embodiments, R⁵ of formula V is a protected hydroxyl group. In certain embodiments, R⁵ of formula V is phosphate. In certain embodiments, R⁵ of formula V is sulfate. In certain other embodiments, R⁵ of formula V is acetate.

In certain embodiments, R⁴ and R⁵ of formula V are both hydrogen. In certain embodiments, only one of R⁴ and R⁵ are hydrogen. In certain embodiments, at least one of R⁴ and R⁵ is hydrogen.

In certain embodiments, compounds of the invention are of the formula:

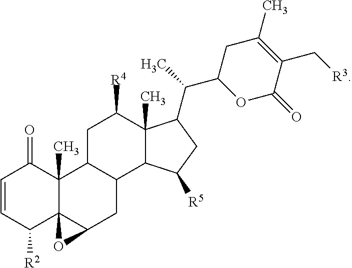

In some embodiments, R² and R³ are —OR^{B}. In some embodiments, R² and R³ are —OH. In some embodiments, R² and R³ are —OAc.

In certain embodiments, compounds of the invention are of the formula:

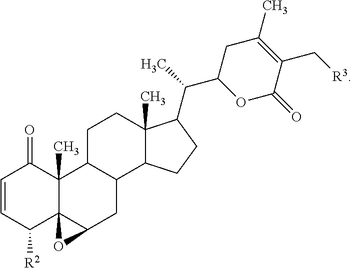

In some embodiments, R² and R³ are —OR^{B}. In some embodiments, R² and R³ are —OH. In some embodiments, R² and R³ are —OAc.

In certain embodiments, compounds of the invention are of the formula:

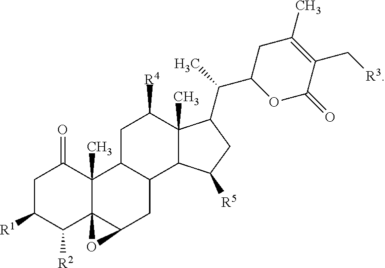

In some embodiments, R¹ is —OSO₃H. In some embodiments, R² and R³ are —OR^{B}. In some embodiments, R² and R³ are independently —OH or —OAc.

In certain embodiments, compounds of the invention are of the formula:

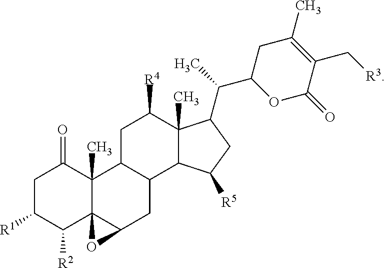

In some embodiments, R¹ is —OSO₃H. In some embodiments, R² and R³ are —OR^{B}. In some embodiments, R² and R³ are independently —OH or —OAc.

Exemplary compounds of the invention include:

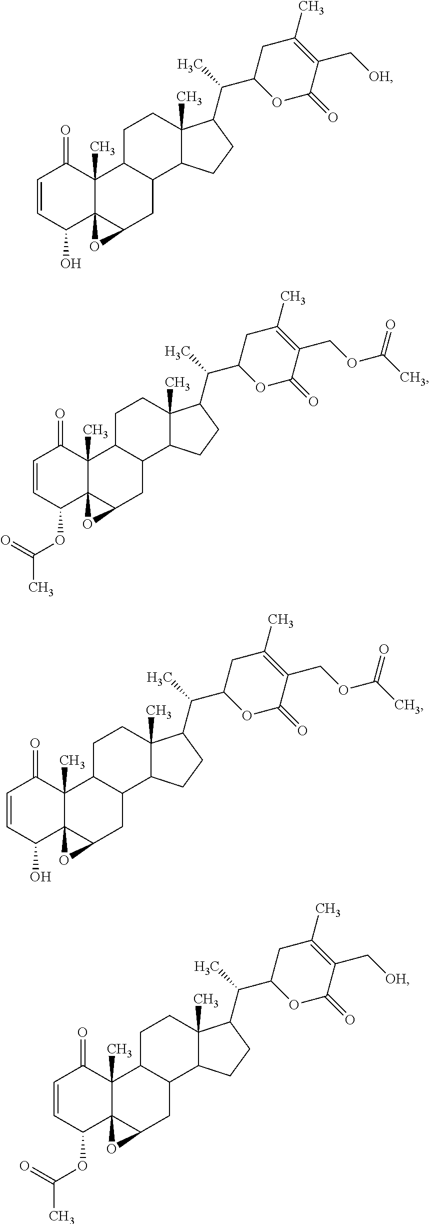

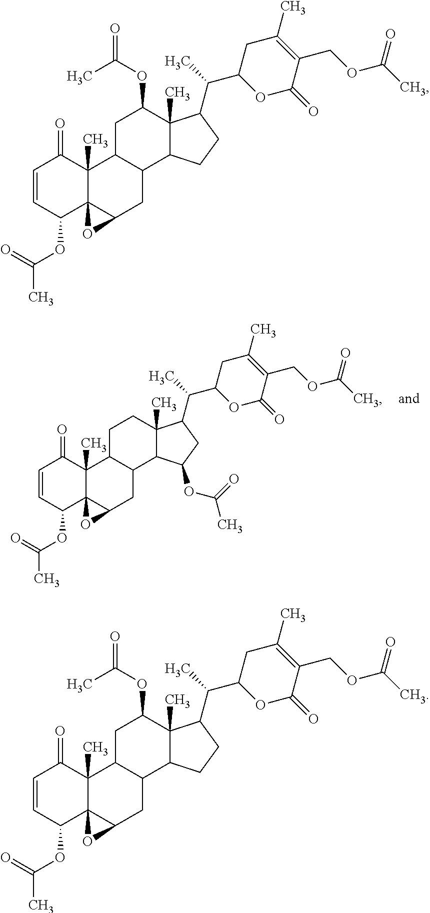

Isolation of Withanolides from W. somnifera

Withanolide natural products are isolated from the aerial tissue and/or roots of W. somnifera. The identity and amounts of natural products isolated is dependent on how the plant is grown. When W. somnifera is grown aeroponically, using chemically-defined nutrient media and without soil, novel natural products can be isolated. In certain embodiments, the amount of a particular natural product may be altered by growing W. somnifera under different conditions.

In certain embodiments, natural products are isolated from W. somnifera which has been grown aeroponically. For example, 2,3-dihydrowithaferin A-3β-O-sulfate may be isolated from aeroponically grown W. somnifera.

In further embodiments, natural products from aeroponically grown W. somnifera are isolated from the aerial tissues of the plant. In further embodiments, natural products from aeroponically grown W. somnifera are isolated from the leaves of the plant. In further embodiments, natural products from aeroponically grown W. somnifera are isolated from the stem of the plant.

In further embodiments, natural products from aeroponically grown W. somnifera are isolated from the roots of the plant.

In certain embodiments, aerial tissues of W. somnifera are extracted with a solvent to give the crude natural product extract. In certain embodiments, the solvent is a polar solvent. In certain embodiments, the solvent is a nonpolar solvent. In certain embodiments, the solvent is a protic solvent. In certain embodiments, the solvent is an aprotic solvent. In certain embodiments, the solvent is a polar, protic solvent. In certain embodiments, the solvent is an alcohol. In certain embodiments, the solvent is methanol. In certain embodiments, the solvent is ethanol. In certain embodiments, the solvent is isopropanol. In certain embodiments, the solvent is a mixture of alcohols. In certain embodiments, the solvent is a mixture of one or more alcohols and water.

In certain embodiments, the crude natural product extract obtained from W. somnifera is purified. In certain embodiments, the extract is purified by chromatography. In certain embodiments, the extract is purified by silica gel chromatography. In certain embodiments, the crude extract is purified by reversed-phase chromatography. In certain embodiments, the crude extract is purified by successive rounds of chromatography. HPLC may be used to purify the desired compounds.

In certain embodiments, the desired natural product is further purified by crystallization.

The purified compounds may be characterized by various analytical methods including elemental analysis, mass spectrometry, IR, UV/vis, NMR, and x-ray crystallography.

Semi-Synthesis of Novel Withanolides from Natural Products

In some embodiments, novel withanolides are synthesized from withanolide natural products. In certain embodiments, novel withanolides are synthesized from withaferin A.

With reference to Scheme 1, epi-withaferin A may be synthesized from withaferin A. Withaferin A may be oxidized according to methods known by those skilled in the art to give 4-dehydrowithaferin A. An appropriate oxidant, for example, is manganese dioxide. 4-Dehydrowithaferin A may then be reduced to give epi-withaferin A. An appropriate reducing agent, for example, is sodium borohydride/cerium trichloride hentahvdrate.

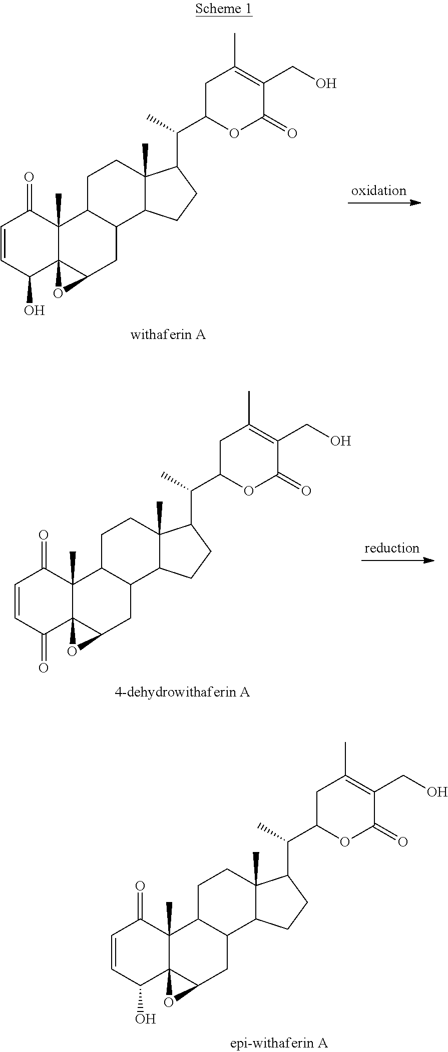

With reference to Scheme 2, 4,27-di-O-acetyl epi-withaferin A may be synthesized from withaferin A using an acetylation procedure. An appropriate acetylating agent, for example, is acetic anhydride.

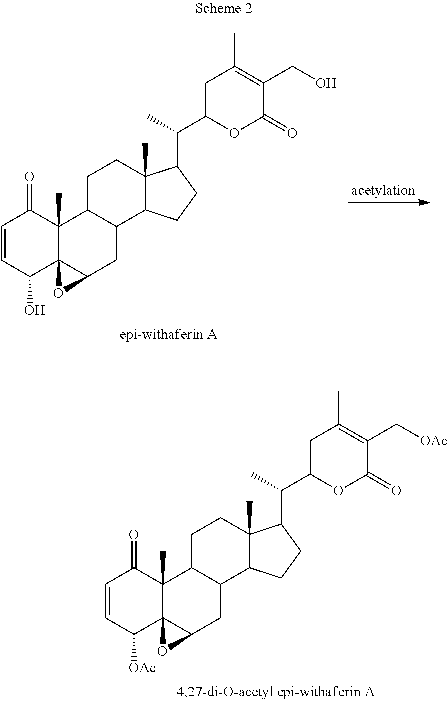

With reference to Scheme 3, 27-O-acetyl epi-withaferin A may be synthesized from withaferin A. Withaferin A may be oxidized to 4-dehydrowithaferin A according to methods described in Scheme 1. 4-Dehydrowithaferin A may be acetylated using methods like those described in Scheme 2 to give 27-O-acetyl-4-dehydrowithaferin A. Reduction of 27-O-acetyl-4-dehydrowithaferin A in a manner analogous to that of Scheme 1 may provide 27-O-acetyl epi-withaferin A.

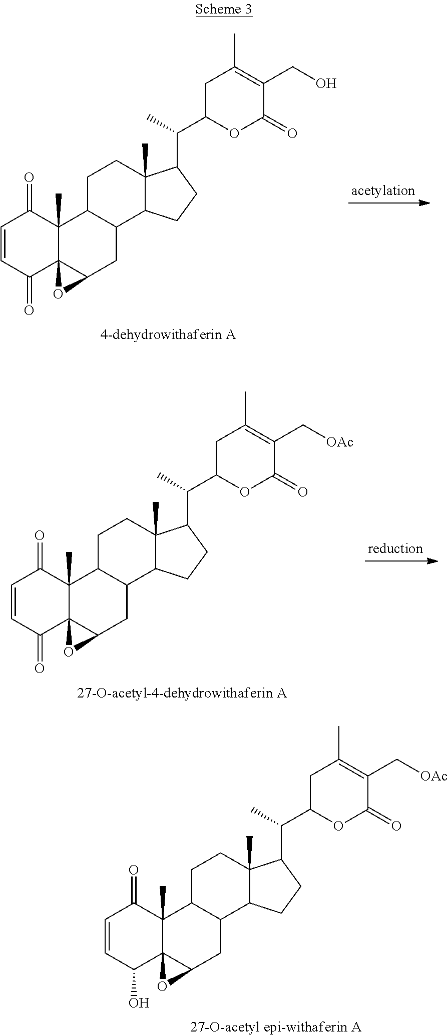

With reference to Scheme 4,4-O-acetyl epi-withaferin A may be synthesized from withaferin A. Withaferin A may be oxidized to 4-dehydrowithaferin A according to methods described in Scheme 1. The 27-hydroxyl group of 4-dehydrowithaferin A may be protected with a protecting group according to methods known to those skilled in the art. Suitable protecting groups include silyl protecting groups (e.g., t-butyldimethylsilyl). 27-O-t-butyldimethylsilyl-4-dehydrowithaferin A may be reduced according to methods analogous to those described in Scheme 1 to give 27-O-t-butyldimethylsilyl epi-withaferin A. 27-O-t-butyldimethylsilyl epi-withaferin A may be acetylated according to methods analogous to those described in Scheme 2 to give 4-O-acetyl-27-O-t-butyldimethylsilyl epi-withaferin A. 4-O-Acetyl-27-O-t-butyldimethylsilyl epi-withaferin A may be deprotected according to methods known to those skilled in the art. A suitable reagent for removing a t-butyldimethylsilyl group, for example, is an aqueous acid. A suitable aqueous acid is, for example, hydrochloric acid.

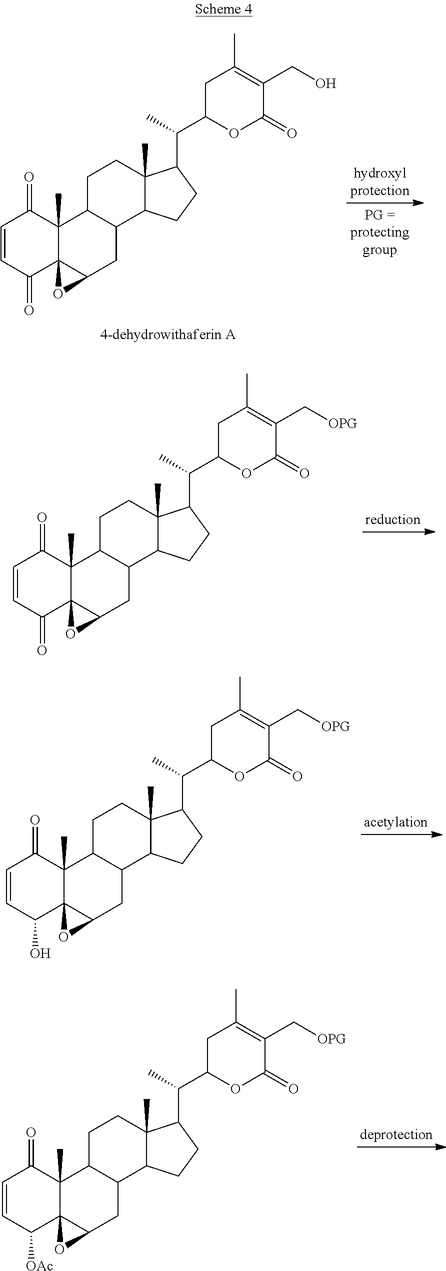

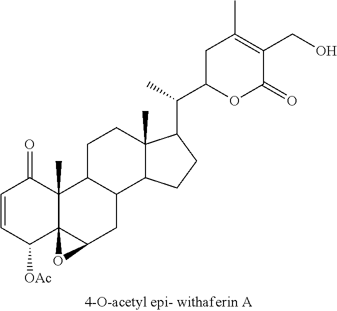

With reference to Scheme 5, 27-O-acetylwithaferin A may be synthesized from withaferin A using methods well known to those skilled in the art. A suitable acetylation procedure, for example, uses acetic anhydride in pyridine.

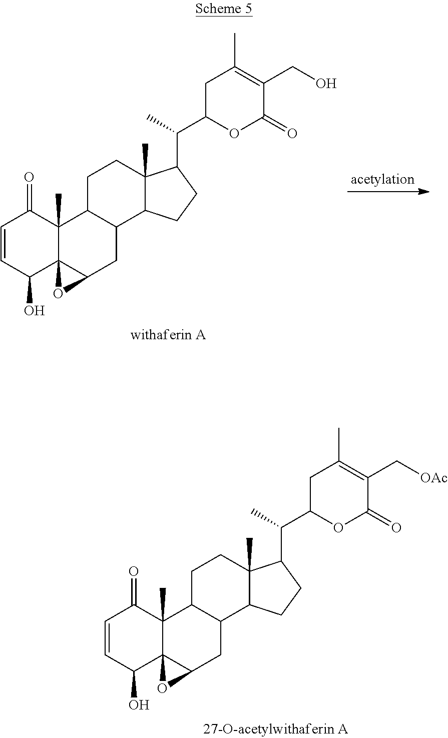

### Anti-Proliferative Activity of Withanolides

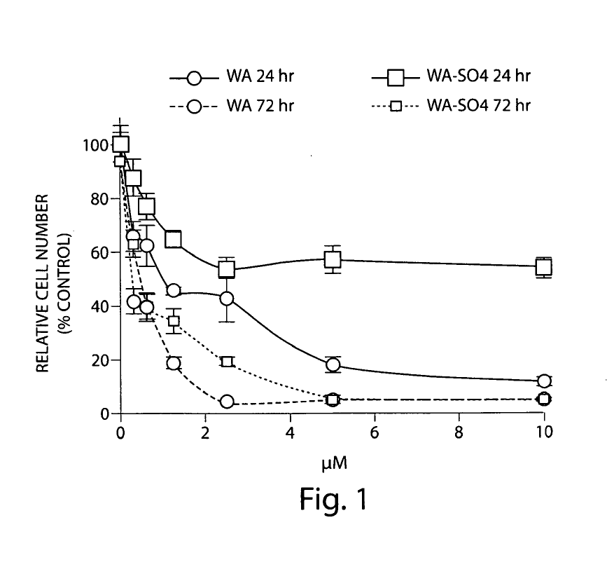

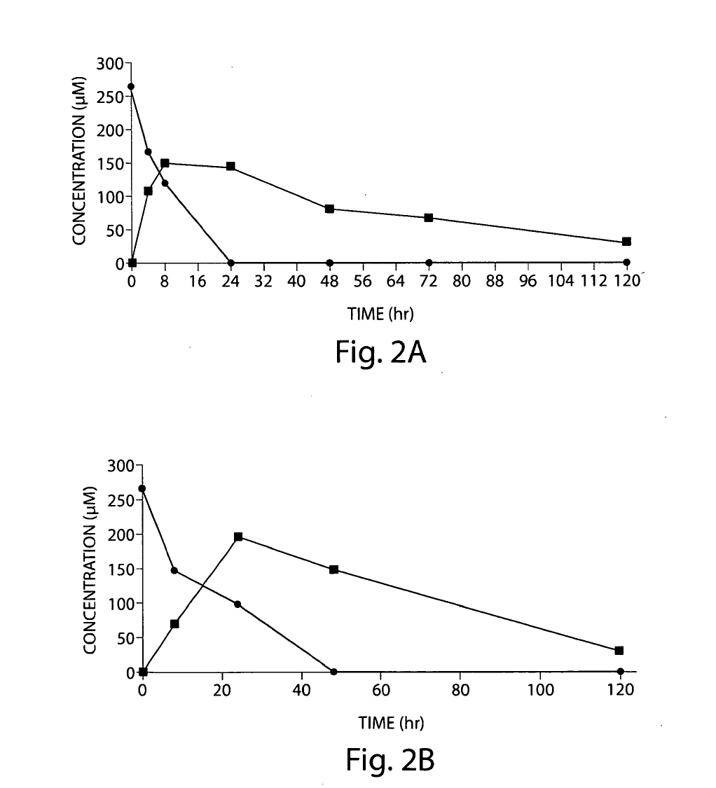

A sulfated withanolide isolated from the aerial tissue of aeroponically-grown W. somnifera, 2,3-dihydrowithaferin A-3β-O-sulfate, displays concentration- and time-dependent inhibition of the proliferation/survival of MCF-7 breast cancer cells (FIG. 1). Withaferin A inhibits the growth of cancer cells at an earlier time point, but after ca. 72 hours dihydrowithaferin A-3β-O-sulfate is equipotent with aferin A. The same results have been reported in two other cancer cell lines (NCI-H460 (non small cell lung) and PC-3M (metastatic prostate cancer)). This phenomenon is likely due to the conversion of dihydrowithaferin A-3β-O-sulfate to withaferin A in the presence of cells, thereby acting as a soluble prodrug of withaferin A. FIG. 2 shows conversion of dihydrowithaferin A-3β-O-sulfate to withaferin A in cell culture media as determined by HPLC.

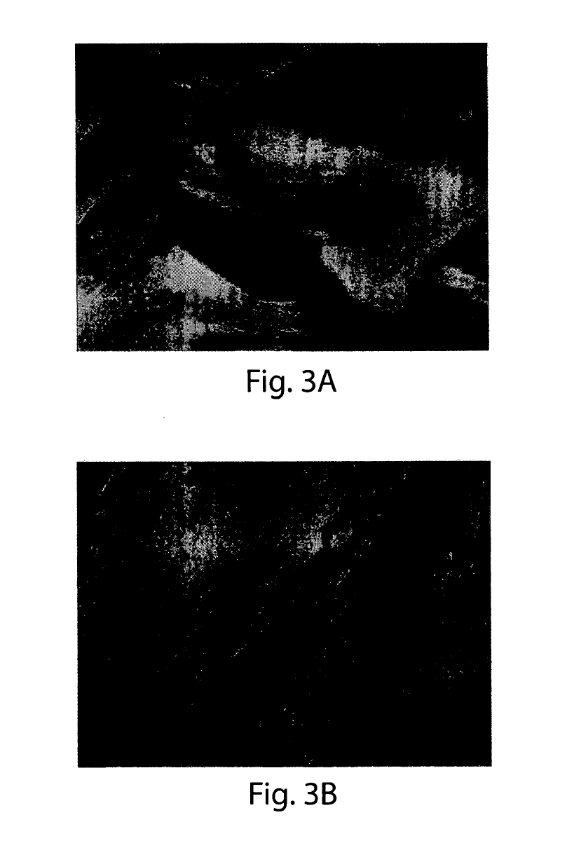

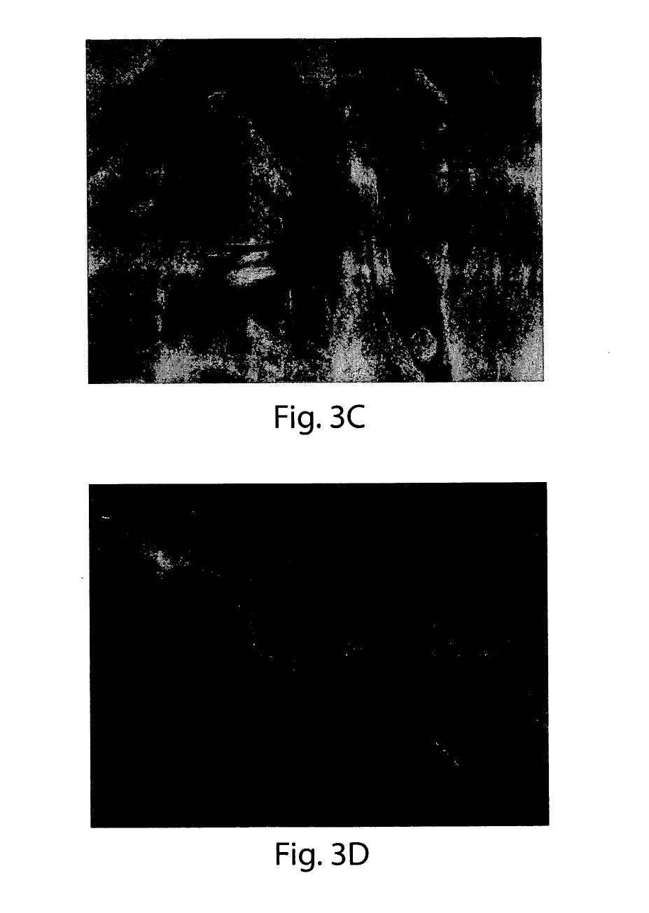

Without wishing to be bound by a particular theory, the possible mode of action for the anti-cancer activity displayed by withanolides is the disruption of cytoskeletal organization with the appearance of focal aggregates of filamentous actin (F-actin) (Falsey, et al., Nat. Chem. Biol. (2006) 2: 33-38, incorporated herein by reference). Human diploid fibroblasts were cultured in the presence of withaferin A (FIGS. 3B and 3D) and dihydrowithaferin A-3β-O-sulfate (FIG. 3C). Both compounds induced F-actin aggregation, but dihydrowithaferin A-3β-O-sulfate required a longer period of time to have this effect.

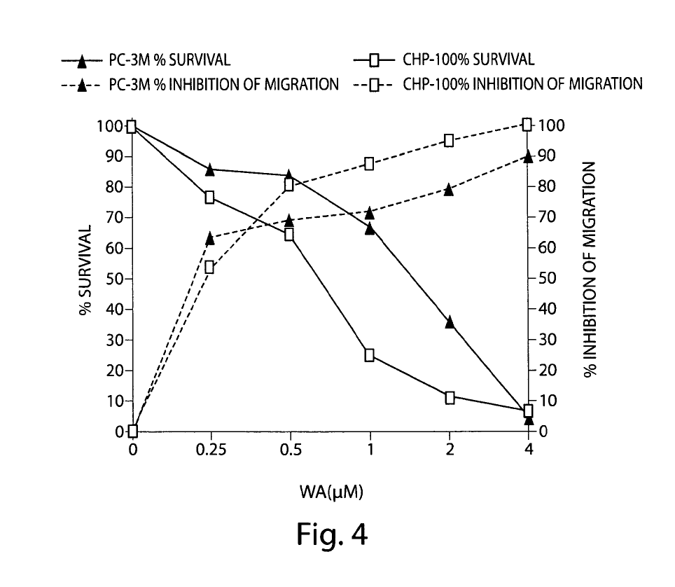

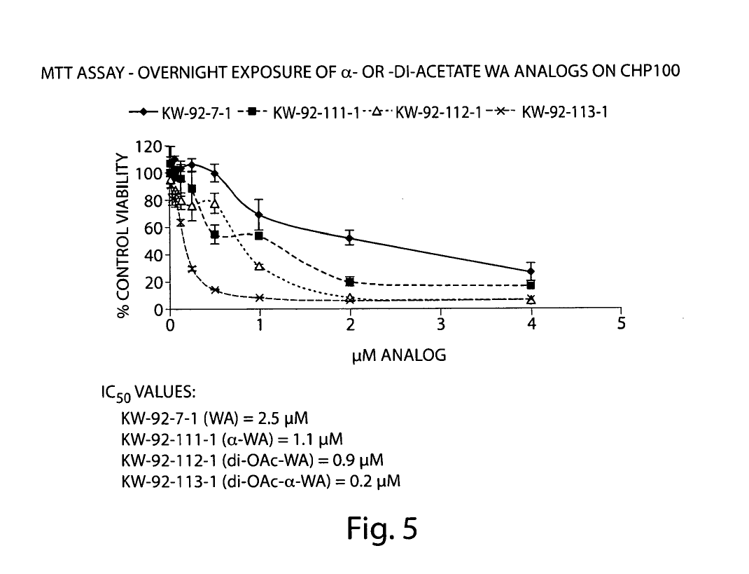

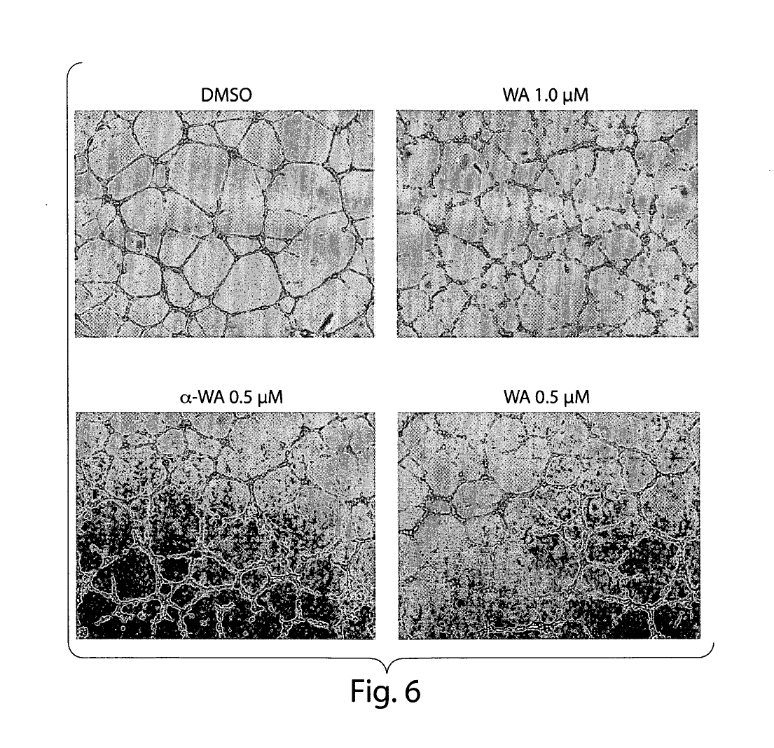

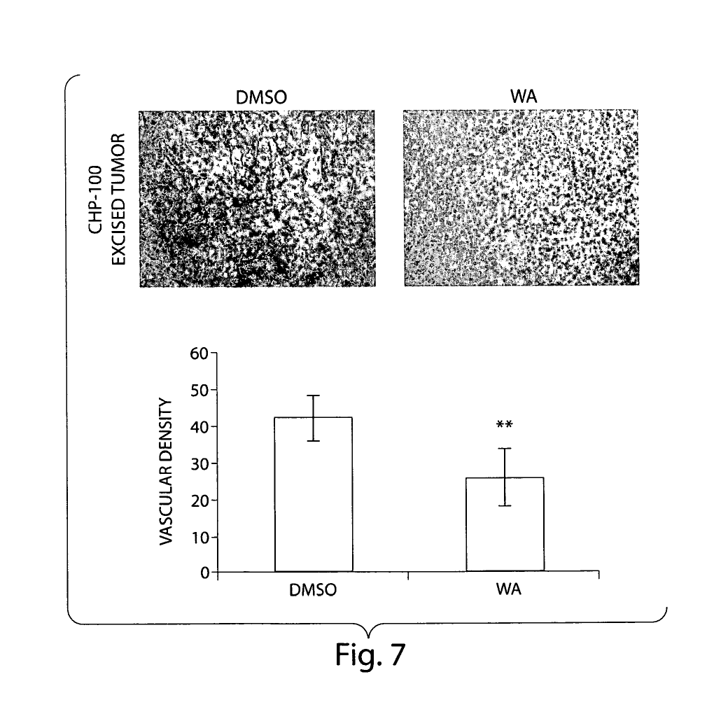

Withaferin A is also shown to inhibit tumor cell migration and invasion in prostate cancer cells and Ewing's sarcoma cells (FIG. 4) and inhibit tumor growth in Ewing's sarcoma (FIG. 5). Withaferin A disrupts the endothelial cell network (FIG. 6) and inhibits tumor vascularization (FIG. 7).

### Withanolide Induction of Heat Shock Response

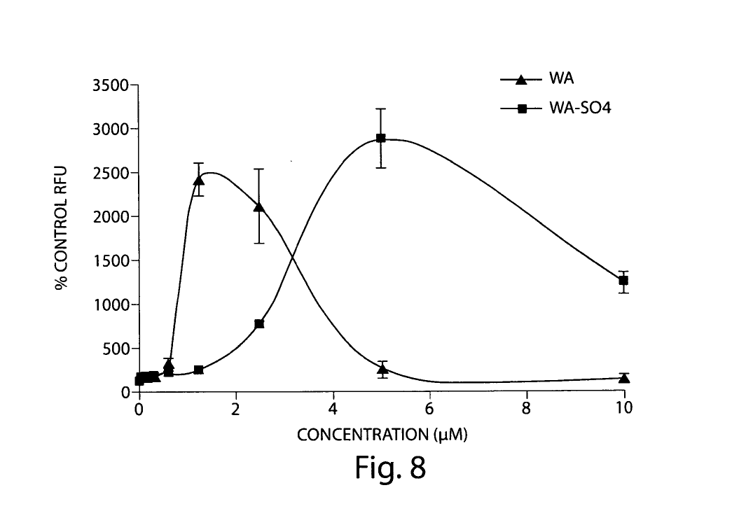

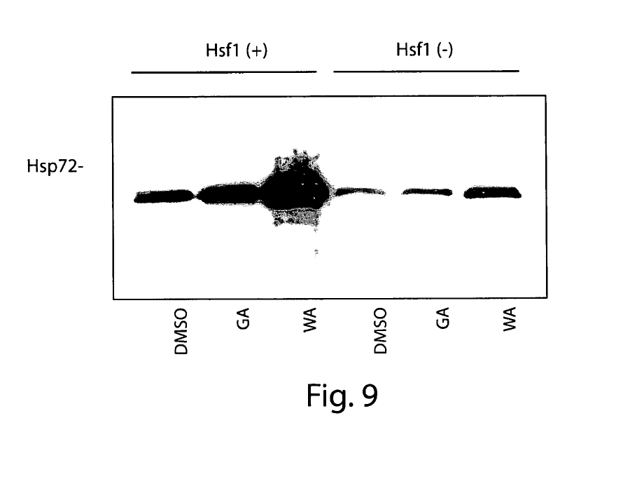

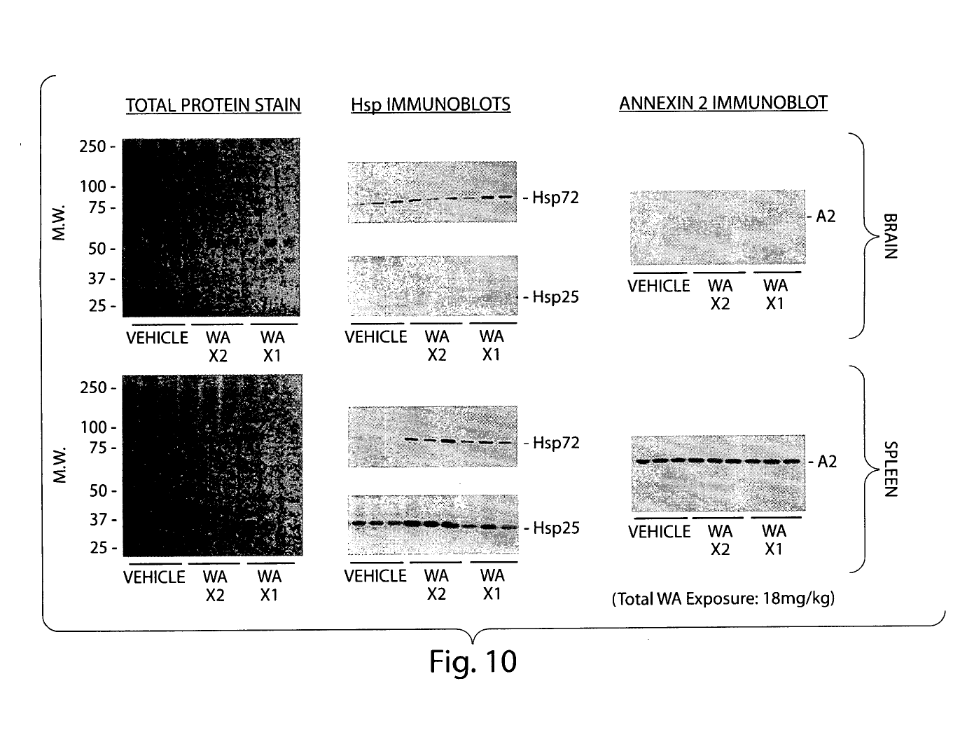

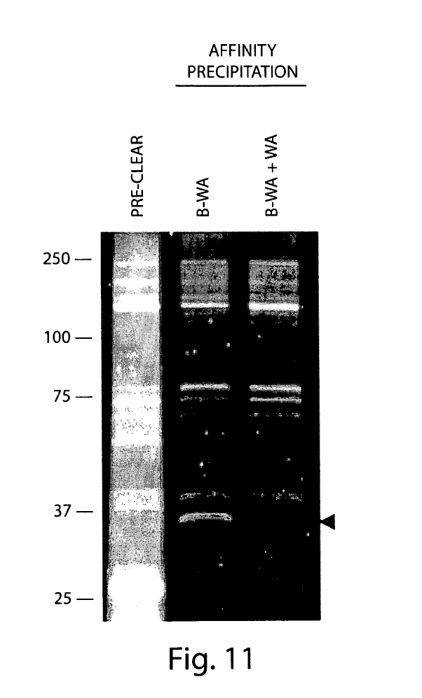

Withaferin A induces a heat shock response in cells, possibly as a consequence of F-actin aggregration as described above. Exposing a heat shock reporter cell line to serial concentrations of withaferin A demonstrated that the response can be induced at compound exposures compatible with cell survival (FIG. 8). The increased heat shock protein expression stimulated by withaferin A requires Heat Shock Factor 1 (HSF1), the dominant transcriptional regulator of the classical response to heat (FIG. 9). Dihydrowithaferin A-3β-O-sulfate also induces a robust heat shock response after overnight treatment of cells, albeit at higher concentrations than that of withaferin A (FIG. 8). Withaferin A induces the heat shock response in spleen cells, where annexin II is present, but not in brain cells where annexin II is absent (FIG. 10). In fact, withaferin A binds stably and selectively to annexin II (FIG. 11), suggesting a role for annexin II in the heat shock response. These results suggest that withanolides and analogs thereof could be useful as therapeutic inducers of the heat shock response, which has been implicated in protection from protein aggregration disorders.

Gene Expression Profiling of Astrocytes Treated with Withaferin A

Primary human astrocytes were exposed to WA (1 μM) or an equal volume of DMSO solvent for 6 hours. RNA was isolated by phenol-chloroform extraction, reverse transcribed, labeled and hybridized to Agilent dual-color human whole genome arrays followed by standard analysis for relative mRNA levels. Results demonstrate induction of an adaptive transcriptional response that includes classic elements of the heat shock response. In addition, it was found that withaferin A also triggers a robust anti-oxidant defense response with marked upregulation of the glutamate-cysteine ligase, the glutamate-cystine transporter, and thioredoxin reductase activity in addition to driving expression of numerous components of neurotrophic pathways.

### Neuroprotective Activity of Withaferin A in a Cell Culture Model of Apoptosis

Neurotrophic factor deprivation-induced apoptosis of rat spinal cord motor neurons was used as a model system to evaluate neuroprotective activity of withaferin A. Primary spinal cord motor neurons were purified from E15 rats. These cells were dissociated from the ventral spinal cord, enriched by density gradient centrifugation, and purified by magnetic bead cell separation using an antibody against p75NTR which is expressed on the cell surface of motor neurons at this developmental age. The resulting cultures are ˜96% motor neurons as assessed by HB9 or islet-1 immunoreactivity and are virtually devoid of astrocytes or microglia. For viability experiments, the cells were plated at a low density (500 cells/well of a 96-well plate). In these cultures, motor neuron survival is highly dependent on trophic factors added to the culture media (in the form of BDNF, GDNF, or cardiotrophin-1), as ˜50% of the attached cells will undergo apoptosis when deprived of trophic factors (Oppenheim, R. W., et al., Nature, 360: 755-757; Henderson, C. E., et al. Science, 266: 1062-1064, 1994). BDNF was used as the trophic factor.

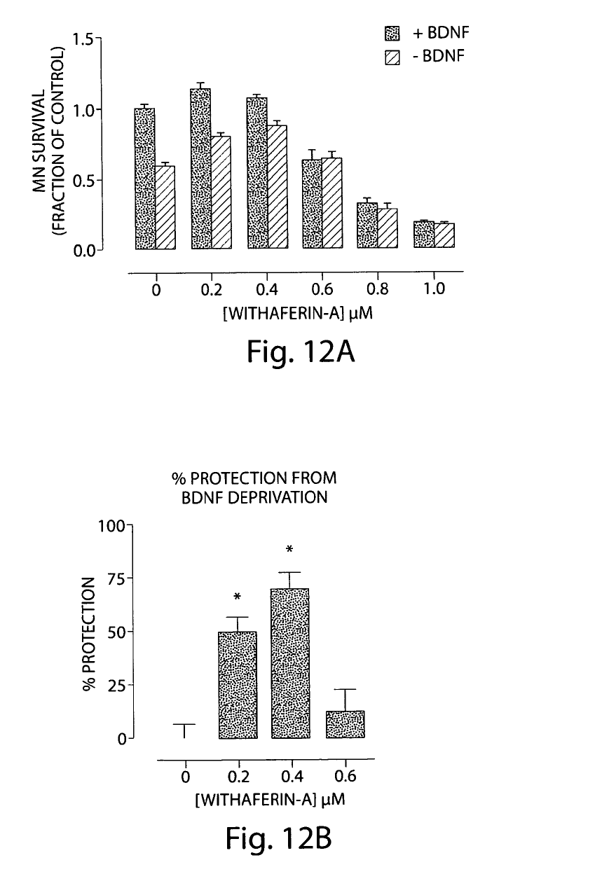

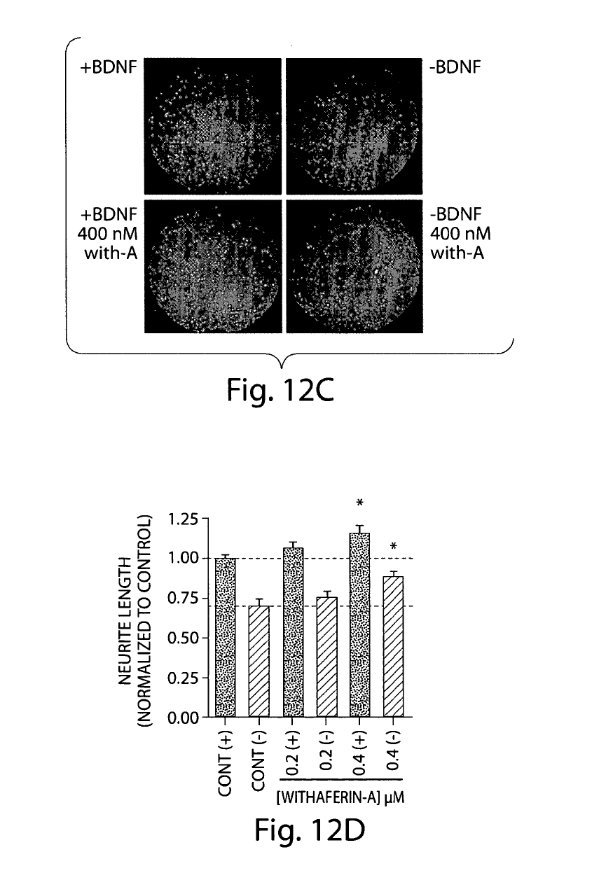

Neuronal survival was assessed by staining with calcein-A, a fluorescent dye that is taken up by viable neurons. Results are presented in FIGS. 12A and 12B. In the absence of BDNF (-BDNF) there was approximately 40% reduction in viable motor neurons compared to wells with BDNF present (+BDNF). Addition of withaferin A (WA) to the motor neuron cultures plated in the absence of BDNF resulted in a 50% reduction in cell death at 200 nM (0.2 μM) concentration of WA and a 75% reduction in cell death at 400 nM (0.4 μM) after 24 hours. This data was acquired using Metamorph software.

In some experiments, a fluorescence image of the entire well within a plate was captured using a flash cytometer (Trophos). Using this method, it is possible to resolve the cell bodies and neurites of surviving motor neurons. FIG. 12C shows results of this assay using WA. In addition to enhancing neuronal survival, WA induces the growth of neurites (FIG. 12D). Analyzing the images with automated image analysis software such as MetaMorph® provides cell counts, neurite number, neurite length, and various other parameters.

### Neuroprotective Effect of WA in a Cell Culture Model of Glutathione Depletion

WA was tested in a second model for neuroprotection that utilizes primary astrocytes cultured from the cerebral cortices of postnatal day 1-3 rat pups. In the CNS, astrocytes play a pivotal role in protecting neurons from oxidative stress, a hallmark of nearly all neurodegenerative diseases and disorders. Astrocytes contain high levels of the major cellular antioxidant glutathione (GSH) and thereby, protect neurons via (i) the release of GSH; and (ii) by providing GSH precursors necessary for neuronal GSH synthesis (for a review, see Dringen et al., Eur. J. Biochem., 267: 4912-4916.). The regulation of GSH synthesis, utilization, and export is reportedly mediated by the transcription factor Nrf2 (Nuclear factor-erythroid 2-related factor 2), which has been found to play a major role in astrocyte-mediated neuronal protection from oxidative stress (Shih et al., J. Neuroscience, 23: 3394-3406, 2003).

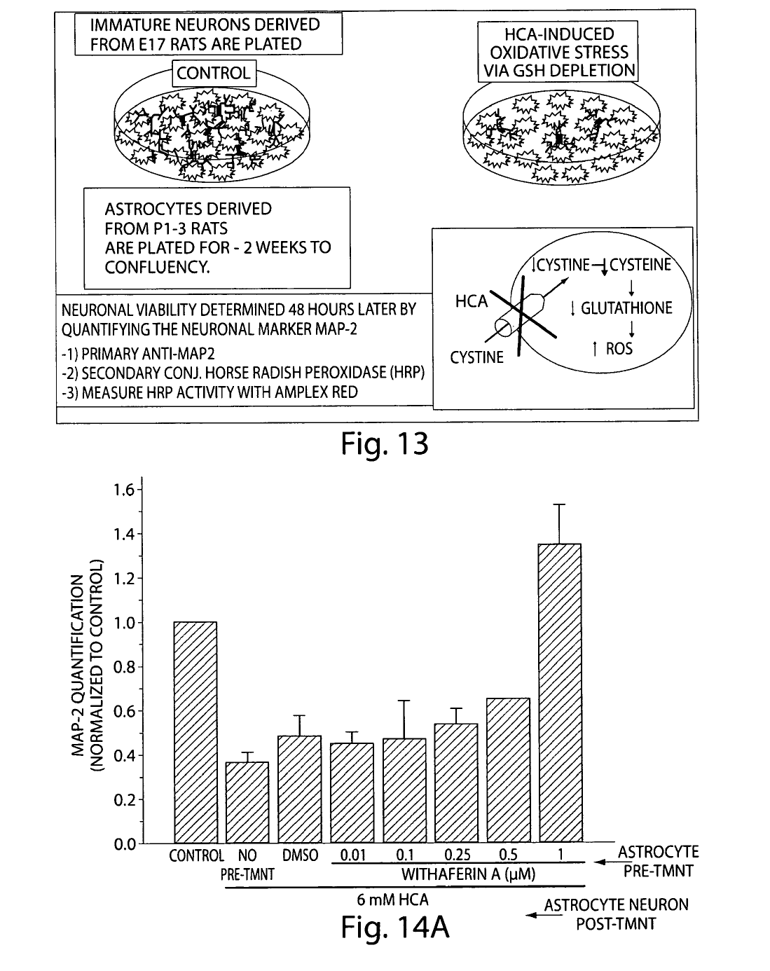

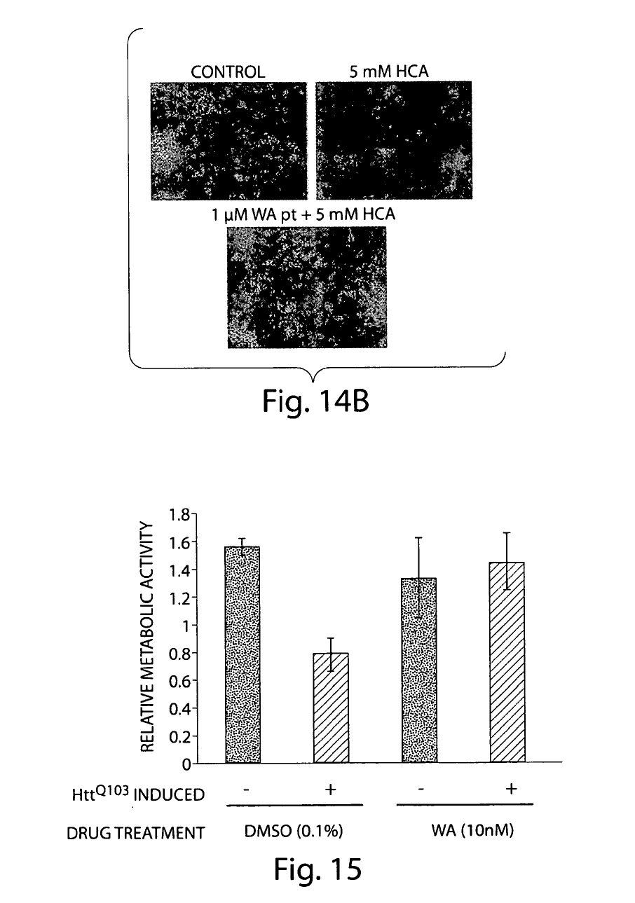

In this assay, which is summarized in FIG. 13, primary astrocytes were isolated from the cerebral cortices of postnatal day 1-3 rat pups, allowed to grow to confluency for ˜2 weeks, and then treated with test compound for 24 h. Following this, the astrocytes were washed twice with serum-containing media to completely remove test compounds from the culture media. Immediately following the washing step, primary neurons derived from embryonic day 17 rat fetuses were plated directly on top of the astrocyte monolayer in the presence or absence of 5 mM homocysteic acid (HCA) (Sigma) to induce oxidative stress-mediated neuronal death. HCA induces oxidative stress by blocking the uptake of cystine, which subsequently decreases intracellular cysteine levels needed for the synthesis of GSH. The resulting decrease in intracellular GSH levels leads to the accumulation of endogenous antioxidants and subsequent oxidative stress-mediated neuronal death (for a review see; Ratan et al., Methods in Enzymology, 352: 183-190, 2002). The concentration of HCA used in this co-culture model induces neuronal death 48 h following treatment, but without induction of astrocytic death. Quantification of the neuron-specific protein microtubule-associated protein-2 (MAP-2) is used to monitor neuronal specific death in this astrocyte-neuron co-culture model as described previously (Carrier et al., J. Neuroscience Methods, 154: 239-244.2006). Briefly, following 48 h HCA treatment the co-culture is fixed with 4% paraformaldehyde for 0.5 h at 37° C. Following wash-out of the fixative, the co-cultures are incubated overnight in a Triton-x 100 containing blocking buffer with primary polyclonal antibodies targeted against MAP-2 (1:500) (Millipore), followed by 0.5 h incubation with rabbit-secondary antibodies conjugated with horse radish peroxidase (HRP) (1:1250) (BioRad). HRP activity is then measured using a reaction buffer containing 150 uM amplex red (Molecular Probes) and 800 uM hydrogen peroxide (Sigma). Increased HRP activity, indicative of higher levels of MAP-2, may thus be quantified spectrophotometrically by monitoring the oxidation byproduct of amplex red, resorufin, which is produced as a consequence of HRP-catalyzed oxidation. Results show that astrocytes pre-treated with aferin A enhance the protection of neurons exposed to oxidative stress via GSH depletion, suggesting that WA increases the antioxidant capacity of the astrocytes. FIGS. 14A and 14B present data showing withaferin A-mediated protection of rat primary neurons from oxidative stress in this assay.

### Effect of WA in PC12 Cell Culture Model of Huntington's Disease (HD)

The PC12 cell culture model of HD originally developed by E. Schweitzer's group (Aiken et al., Neurobiol Dis, 16: 546-555, 2004) was used for evaluation of withaferin A. As shown in FIG. 15, a low nanomolar concentration of WA effectively rescued toxicity in this model. Concentrations of WA up to 10 μM did not significantly impair MTT reduction by PC12 cells demonstrating a wide margin between beneficial and toxic effects for WA in this assay system (data not shown).

### Uses of Withaferin A Analogs and Pharmaceutical Compositions Thereof

The invention further provides methods of treating a disease using an analog of withaferin A. The inventive method involves the administration of a therapeutically effective amount of an inventive compound to a subject (including, but not limited to a human or other animal) in need of it.

Certain inventive compounds activate the heat shock network. Thus, in certain embodiments, the present invention provides a method for treating a heat shock network-associated disorder comprising the step of administering to a patient in need thereof a compound of the present invention or pharmaceutically acceptable composition thereof.

As used herein, the term “heat shock network-associated” disorders means any disease or other deleterious condition in which the heat shock network is known to play a role. Accordingly, another embodiment of the present invention relates to treating or lessening the severity of one or more diseases in which the heat shock network is known to play a role including, but not limited to, autoimmune diseases as well as Huntington's disease, Parkinson's disease, Alzheimer's disease, and other disorders associated with protein misfolding and/or aggregation.

Certain inventive compounds alter the actin bundling activity of annexin II. Thus, in certain embodiments, the present invention provides a method for treating an annexin II-mediated disorder comprising the step of administering to a patient in need thereof a compound of the present invention or pharmaceutically acceptable composition thereof.

As used herein, the term “annexin II-mediated” disorders means any disease or other deleterious condition in which annexin II is known to play a role. Accordingly, another embodiment of the present invention relates to treating or lessening the severity of one or more diseases in which annexin II is known to play a role including, but not limited to, atherosclerosis, diabetes, disorders associated with pathological proliferation of blood vessels such as diabetic retinopathy, macular degeneration, and cancers, e.g., glioma, colorectal carcinoma, gastric carcinoma, hepatic carcinoma, small cell lung carcinoma, and pancreatic carcinoma.

Certain inventive compounds inhibit the 20S proteasome. Thus, in certain embodiments, the present invention provides a method for treating a 20S proteasome-mediated disorder comprising the step of administering to a patient in need thereof a compound of the present invention or pharmaceutically acceptable composition thereof.

As used herein, the term “20S proteasome-mediated” disorders means any disease or other deleterious condition in which the 20S proteasome is known to play a role. Accordingly, another embodiment of the present invention relates to treating or lessening the severity of one or more diseases in which the 20S proteasome is known to play a role including, but not limited to, multiple myeloma, pancreatic cancers, B-cell related cancers such as non-Hodgkin's lymphoma, glioma, and autoimmune diseases.

Certain inventive compounds inhibit the intermediate filament protein vimentin. Thus, in certain embodiments, the present invention provides a method for treating a vimentin-mediated disorder comprising the step of administering to a patient in need thereof a compound of the present invention or pharmaceutically acceptable composition thereof.

As used herein, the term “vimentin-mediated” disorders means any disease or other deleterious condition in which vimentin is known to play a role. Accordingly, another embodiment of the present invention relates to treating or lessening the severity of one or more diseases in which vimentin is known to play a role including, but not limited to, autoimmune diseases, organ transplantation, vascular disease, and giant axonal neuropathy.

Certain inventive compounds inhibit NFκB activation. Thus, in certain embodiments, the present invention provides a method for treating NFκB-mediated disorders comprising the step of administering to a patient in need thereof a compound of the present invention or pharmaceutically acceptable composition thereof.

As used herein, the term “NFκB-mediated” disorders means any disease or other deleterious condition in which NFκB is known to play a role. Accordingly, another embodiment of the present invention relates to treating or lessening the severity of one or more diseases in which NFκB activation is known to play a role including, but not limited to, rheumatoid arthritis, inflammatory bowel disease, asthma and other inflammatory disorders, as well as cancers such as leukemia, lymphoma, colon cancer, and ovarian cancer.

Certain inventive compounds inhibit protein kinase C (PKC). Thus, in certain embodiments, the present invention provides a method for treating a PKC-mediated disorder comprising the step of administering to a patient in need thereof a compound of the present invention or pharmaceutically acceptable composition thereof.

As used herein, the term “PKC-mediated” disorders means any disease or other deleterious condition in which PKC is known to play a role. Accordingly, another embodiment of the present invention relates to treating or lessening the severity of one or more diseases in which PKC is known to play a role including, but not limited to, Alzheimer's disease, diabetic vascular disease, glaucoma, lung cancer, colon cancer, renal cell cancer, hepatocellular cancer, prostate cancer, ovarian cancer, bladder cancer, and brain cancer.

Certain inventive compounds induce apoptosis, particularly Par-4-dependent apoptosis. Thus, in certain embodiments, the present invention provides a method for treating a Par-4-mediated disorder comprising the step of administering to a patient in need thereof a compound of the present invention or pharmaceutically acceptable composition thereof.

As used herein, the term “Par-4-mediated” disorders or conditions means any disease or other deleterious condition in which Par-4 is known to play a role. Accordingly, another embodiment of the present invention relates to treating or lessening the severity of one or more diseases in which apoptosis is known to play a role including, but not limited to, autoimmune diseases and cancer.

The compounds and pharmaceutical compositions of the present invention may be used in treating or preventing any disease or condition including, but not limited to, asthma, arthritis, inflammatory diseases (e.g., Crohn's disease, rheumatoid arthritis, psoriasis), proliferative diseases (e.g., cancer, benign neoplasms, diabetic retinopathy), cardiovascular diseases, neurodegenerative diseases, protein aggregation disorders (e.g., Huntington's disease, Alzheimer's disease), and autoimmune diseases (e.g., rheumatoid arthritis, lupus). The inventive compounds and pharmaceutical compositions may be administered to animals, preferably mammals (e.g., domesticated animals, cats, dogs, mice, rats), and more preferably humans. Any method of administration may be used to deliver the inventive compound or pharmaceutical composition to the animal. In certain embodiments, the compound or pharmaceutical composition is administered orally. In other embodiments, the compound or pharmaceutical composition is administered parenterally.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The invention further relates to a method for treating, ameliorating, or preventing cellular neoplasia by administration of an effective amount of a compound according to this invention to a mammal, in particular a human in need of such treatment. A “neoplasia” is defined by cells displaying aberrant cell proliferation and/or survival and/or a block in differentiation. The term “neoplasia” includes benign neoplasia, which is described by hyperproliferation of cells, incapable of forming an aggressive, metastasizing tumor in vivo, and, in contrast, malignant neoplasia, which is described by cells with multiple cellular and biochemical abnormalities, capable of forming a systemic disease, for example forming tumor metastases in distant organs.

Compounds according to this invention can be particularly used for the treatment of malignant neoplasia, also described as cancer, characterized by tumor cells finally metastasizing into distinct organs or tissues. Examples of malignant neoplasia treated with compounds according to the present invention include solid and hematological tumors. Solid tumors are exemplified by tumors of the breast, bladder, bone, brain, central and peripheral nervous system, colon, connective tissue, endocrine glands (e.g., thyroid and adrenal cortex), esophagus, endometrium, germ cells, head and neck, kidney, liver, lung, larynx and hypopharynx, mesothelioma, muscle, ovary, pancreas, prostate, rectum, renal, small intestine, soft tissue, testis, stomach, skin, ureter, vagina, and vulva. Malignant neoplasia include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), chronic lymphocytic leukemia (CLL), Hodgkins disease, multiple myeloma, and T-cell lymphoma. Also included are myelodysplastic syndrome, plasma cell neoplasia, paraneoplastic syndromes, cancers of unknown primary site as well as AIDS-related malignancies.

It will also be appreciated that a cancer (malignant neoplasia) as a life-threatening disease process does not necessarily require the formation of metastases in distant organs. Certain tumors exert devastating effects on the primary organ itself through their aggressive growth properties. These can lead to the destruction of the tissue and organ structure finally resulting in failure of the assigned organ function.

In certain embodiments, the current invention provides a method for the treatment of benign neoplasia. Examples of benign neoplasia treated with compounds according to the present invention include, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In certain embodiments, the present invention provides methods for treating or lessening the severity of autoimmune diseases including, but not limited to, inflammatory bowel disease, arthritis, systemic lupus erythematosus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

In some embodiments, the present invention provides a method for treating or lessening the severity of one or more diseases and conditions, wherein the disease or condition is selected from heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the present invention provides a method for treating or lessening the severity of an inflammatory disease including, but not limited to, asthma, appendicitis, Behcet's disease, Blau syndrome, blepharitis, bronchiolitis, bronchitis, bursitis, cervicitis, cholangitis, cholecystitis, chronic recurrent multifocal osteomyelitis (CRMO), colitis, conjunctivitis, cryopyrin associated periodic syndrome (CAPS), cystitis, dacryoadenitis, dermatitis, dermatomyositis, encephalitis, endocarditis, endometritis, enteritis, enterocolitis, epicondylitis, epididymitis, familial cold-induced autoinflammatory syndrome, familial Mediterranean fever (FMF), fasciitis, fibrositis, gastritis, gastroenteritis, hepatitis, hidradenitis suppurativa, laryngitis, mastitis, meningitis, mevalonate kinase deficiency (MKD), Muckle-Well syndrome, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, pyoderma gangrenosum and acne syndrome (PAPA), pyogenic sterile arthritis, rhinitis, salpingitis, sinusitis, stomatitis, synovitis, systemic juvenile rheumatoid arthritis, tendonitis, TNF receptor associated periodic syndrome (TRAPS), tonsillitis, uveitis, vaginitis, vasculitis, or vulvitis.

In certain embodiments, the present invention provides methods for treating or lessening the severity of arthropathies and osteopathological diseases including, but not limited to, rheumatoid arthritis, osteoarthrtis, gout, polyarthritis, and psoriatic arthritis.

In certain embodiments, the present invention provides methods for treating or lessening the severity of hyperproliferative diseases including, but not limited to, psoriasis or smooth muscle cell proliferation including vascular proliferative disorders, atherosclerosis, and restenosis. In certain embodiments, the present invention provides methods for treating or lessening the severity of endometriosis, uterine fibroids, endometrial hyperplasia and benign prostate hyperplasia.

In certain embodiments, the present invention provides methods for treating or lessening the severity of acute and chronic inflammatory diseases and dermal diseases including, but not limited to, ulcerative colitis, inflammatory bowel disease, Crohns disease, allergic rhinitis, allergic dermatitis, cystic fibrosis, chronic obstructive bronchitis, and asthma.

In some embodiments, the present invention provides a method for treating or lessening the severity of a cardiovascular disorder including, but not limited to, myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis, ischemic stroke, cardiac hypertrophy and heart failure.

In certain embodiments, the present invention provides methods for treating or lessening the severity of neuropathological disorders and/or protein aggregation disorders including, but not limited to, Parkinson's disease, Alzheimer's disease or polyglutamine related disorders including, but not limited to, Huntington's disease, Spinocerebellar ataxia 1 (SCA 1), Machado-Joseph disease (MJD)/Spinocerebella ataxia 3 (SCA 3), Kennedy disease/Spinal and bulbar muscular atrophy (SBMA), Dentatorubral pallidolusyian atrophy (DRPLA), fronto-temporal dementia, Lewy body disease, Pick's disease, and progressive supranuclear palsy (PSP).

In some embodiments, the invention provides methods of treating a subject in need of neuroprotection. In some embodiments, the subject has suffered a stroke, seizure, or traumatic injury to the nervous system or has suffered exposure to a toxic agent, e.g., a neurotoxic agent. For example, in some embodiments the subject has suffered a spinal cord injury. In some embodiments, the subject has suffered or is expected to suffer oxidative stress to the nervous system or a portion thereof (e.g., the central nervous system (CNS) or a portion thereof (e.g., brain, brain region, spinal cord)), or the peripheral nervous system (PNS) or a portion thereof, such as one or more nerves or nerve trunks. In some embodiments, said nerve is a cranial nerve. In some embodiments said oxidative stress is caused at least in part by exposure of the subject to a toxic agent, e.g., a neurotoxin. In some embodiments, the toxic agent is a chemical compound. A chemical compound can be, e.g., a polypeptide, nucleic acid, small organic molecule, etc. A chemical compound can be invented by man or can be a naturally occurring compound. In some embodiments, the toxic agent is an infectious agent or a substance produced by an infectious agent (e.g., a bacterium) or encoded in its genome. In some embodiments the toxic agent is a virus, e.g., a neurotropic virus. In some embodiments the subject has suffered or is expected to suffer an event that causes oxygen deprivation, nutrient (e.g., glucose) deprivation, and/or growth factor deprivation of nervous system cells. In some embodiments the subject has suffered a hemorrhagic event in the nervous system, e.g., a hemorrhagic stroke, subarachnoid hemorrhage, or aneurysm. In some embodiments a subject suffers from or is at increased risk of (e.g., has one or more art-recognized risk factors for) a disease or condition characterized by neuronal deterioration or loss, e.g., a neuropathy. In some embodiments the subject suffers or is at increased risk of diabetes (e.g., diabetic neuropathy), motor neuron disease, or glaucoma. In some embodiments, administering a compound of the invention inhibits at least some death (e.g., apoptosis) and/or deterioration of nervous system cells that would otherwise occur, e.g., the invention protects at least some nervous system cells from undergoing death or deterioration. In some embodiments, said nervous system cells comprise neuronal cells (also termed “neurons”). A neuronal cell is often characterized, at least in part, by containing one or more markers of neuronal differentiation. Such a marker can be, for example, a neurofilament (e.g., heavy (NF-H), medium (NF-M) or light neurofilament (NF-L) proteins, nestin and α-internexin) NeuN, or MAP2. A neuronal cell further is often characterized as having one or more cell processes (e.g., axon, dendrite). In some embodiments, said nervous system cells comprise glial cells, e.g., astrocytes, oligodendrocytes, and/or microglia. Without wishing to be bound by theory, such non-neuronal nervous system cells may secrete neurotrophic factors or otherwise promote survival and/or inhibit deterioration of neuronal cells. For example, such cells, e.g., astrocytes, may secrete one or more anti-oxidants or anti-oxidant precursors. In some embodiments, the invention provides a method of providing an acute neuroprotective effect by administering a compound of the invention close to the time of acute nervous system insult (e.g., stroke, seizure, injury, toxin exposure), thereby producing an acute neuroprotective effect in at least some neuronal cells. In some embodiments said administration occurs prior to, e.g., within 2 hours, 4 hours, or 6 hours prior to occurrence of the insult. In some embodiments said administration occurs within 24 hours or within 48 hours prior to occurrence of the insult. For example, a compound may be administered before a surgical procedure that is expected to result in neuronal damage, oxygen or nutrient deprivation, or otherwise have deleterious effects on the nervous system and/or before administration of a therapeutic agent that may have such an effect (e.g., as an undesired “side effect”). In some embodiments said administration occurs subsequent to, e.g., within 2 hours, 4 hours, or 6 hours after occurrence of the insult. In some embodiments, said administration occurs within 24 hours or within 48 hours after occurrence of the insult. In some embodiments administration occurs chronically, e.g., the compound is administered multiple times (or continuously) over a time period of at least 6 weeks, e.g., a period of at least 6 weeks after occurrence of the insult. In some embodiments, a neuroprotective effect is evident within 24 hours, or within 48 hours, after administration of a compound, e.g., the extent of neuronal death or deterioration is reduced relative to what would be expected had the compound not been administered. In some embodiments, neuronal death, e.g., apoptosis, is reduced by at least 20%, e.g., by between 20% and 90%, e.g., by between 40% and 80%, e.g., by between 50% and 75%. If desired, cell viability and/or apoptosis may be assessed using a variety of assays known in the art. In some embodiments, neuroprotection according to the inventive methods results in an improved functional outcome relative to what would be otherwise expected (e.g., relative to a control). In some embodiments, the invention provides a method of inhibiting neuronal excitotoxicity, e.g., excitotoxicity induced by an excitatory amino acid such as NMDA or glutamate (e.g., an abnormally elevated level or sudden release of large amounts of such amino acid(s)). In some embodiments, the invention provides a method of inhibiting ischemic reperfusion injury. In some embodiments, a compound according to the invention provides a neurotrophic effect, e.g., promotes survival, development, and/or growth of neurons. In some embodiments, a compound according to the invention has an effect that at least in part mimics that of nerve growth factor (NGF), brain-derived neurotrophic factor (BDNF), ciliary neurotrophic factor (CNTF), neurotrophin-3 (NT-3), erythropoietin (EPO), and/or neurotrophin-4 (NT-4). In some embodiments, a compound according to the invention augments a deficiency of at least one of said neurotrophic factors and/or is administered together with one or more of said neurotrophic factors. In some embodiments, the invention provides a method of promoting neurite outgrowth and/or axonal outgrowth. In some embodiments, said promoting of neurite outgrowth and/or axonal outgrowth occurs in neurons that have been subjected to an injury that results in severing of an axon. In some embodiments, said promoting of neurite outgrowth and/or axonal outgrowth occurs in neurons that are at least in part deprived of a neurotrophic factor, e.g., BDNF-deprived. In some embodiments, the invention provides a method of enhancing peripheral axon and/or nerve regeneration, e.g., after a crush injury.

The present invention further includes a method for the treatment of mammals, including humans, which are suffering from one of the above-mentioned conditions, illnesses, disorders, or diseases. The method comprises that a pharmacologically active and therapeutically effective amount of one or more of the compounds according to this invention, which function to induce various cellular effects, induce the heat shock response, arrest cell proliferation, induce cell differentiation, and/or induce apoptosis, is administered to the subject in need of such treatment.

The invention further relates to the use of the compounds according to the present invention for the production of pharmaceutical compositions which are employed for the treatment and/or prophylaxis and/or amelioration of the diseases, disorders, illnesses, and/or conditions as mentioned herein.

The invention further relates to the use of the compounds according to the present invention for the production of pharmaceutical compositions that activate the heat shock response.

The invention further relates to the use of the compounds according to the present invention for the production of pharmaceutical compositions for inhibiting or treating cellular neoplasia, such as benign or malignant neoplasia, e.g., cancer.

The invention further relates to the use of the compounds according to the present invention for the production of pharmaceutical compositions which can be used for treating, preventing, or ameliorating of diseases responsive to arresting aberrant cell growth, such as proliferative diseases of benign or malignant behavior, such as any of those diseases mentioned herein.

The invention further relates to the use of the compounds according to the present invention for the production of pharmaceutical compositions which can be used for treating, preventing, or ameliorating of disorders responsive to induction of apoptosis, such as any of those diseases mentioned herein.

The exact amount required will vary from subject to subject, depending on the species, age, and general condition of the subject, the particular compound, its mode of administration, its mode of activity, and the like. The compounds of the invention are preferably formulated in dosage unit form for ease of administration and uniformity of dosage. It will be understood, however, that the total daily usage of the proteins and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific protein employed; the specific composition employed; the age, body weight, general health, sex, and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the proteins of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, e.g., for treating cancer and/or when a pro-apoptotic effect is desired, a dose that is at or relatively close to the maximum tolerated dose (MTD) is used. In some embodiments, a dose between 50% and 100% of MTD may be used. In some embodiments, a dose between 75% and 100% of MTD may be used. In some embodiments, e.g., in methods of treating a neurodegenerative disease, providing neuroprotection, and/or promoting axonal and/or neurite outgrowth, a lower dose is used than in methods for treating cancer. In some embodiments, the dose for use in such methods is between 10- and 100-fold lower than the MTD and/or between 10- and 100-fold lower than the dose used in cancer. MTD can be determined using standard methods known to those skilled in the art.

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as poly(lactide-co-glycolide). Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a method of local administration to the nervous system or a portion thereof is used to administer a compound according to the invention. In some embodiments a compound according to the invention is administered using an internal (implantable) or external pump system to deliver a compound according to the invention to the CNS. Such systems can comprise a reservoir from which continuous or intermittent release of a composition occurs into the target tissue or in the vicinity thereof, e.g., via a catheter. The pump may be programmed to release predetermined amounts at predetermined time intervals. See, e.g., U.S. Pat. No. 6,263,237, which is incorporated herein by reference. In some embodiments a technique of regional delivery of therapeutic agents directly into brain parenchyma, such as intracerebral microinfusion, is used. In certain embodiments delivery is accomplished by surgically implanting a catheter through the skull so that the tip has access to a CSF-containing space. The other end of the catheter is then connected to a reservoir (e.g., an Ommaya reservoir), which is placed beneath the scalp (subcutaneously). Methods for administering agents to the spinal cord, e.g., methods such as are commonly used in the treatment of chronic pain to deliver analgesic agents (e.g., intrathecal administration such as by injection) may be used in certain embodiments of the invention. If a pump is used, the catheter may be implanted so that the discharge portion lies in the intrathecal space while the other end is connected to the pump reservoir.

For local administration to the PNS, if desired, injection or infiltration into a nerve or nerve trunk, e.g., adjacent to a site of nerve damage or injury, may be used. Methods for administering anesthetic agents to diverse nerves, nerve bundles, etc., within the PNS are well known in the art, and are of use in various embodiments of the invention.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. For example, an inventive compound may be administered concurrently with another anticancer agent and/or with radiation in order to treat cancer. In some embodiments an inventive compound is administered concurrently with another neuroprotective agent in order to treat a subject in need of neuroprotection and/or concurrently with a procedure or process such as inducing hypothermia or hyperbaric oxygen treatment. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

These and other aspects of the present invention will be further appreciated upon consideration of the following Examples, which are intended to illustrate certain particular embodiments of the invention but are not intended to limit its scope, as defined by the claims.

### EXAMPLES

### General Experimental Procedures

Reagents and solvents for extraction and chemical reactions were purchased from Aldrich Chemical Co. Bakerbond C₁₈ (40 μM) was a product of J. T. Baker Inc. Kromasil C₁₈ reversed phase column (250×4.6 mm, 5 μm) for HPLC was obtained from Supelco Inc. Melting point was determined on an electrothermal melting point apparatus and is not corrected. Optical rotation was measured with JASCO Dip-370 polarimeter. IR spectrum was for KBr disk recorded on a Shimadzu FTIR-8300 spectrometer. UV was recorded with a Shimadzu UV-1601 spectrophotometer. ¹H NMR and ¹³C NMR spectra were measured on a Bruker DRX-500. Mass spectra were recorded on a Shimadzu LCMS QP8000α and an IonSpec FT mass spectrometer (for HRMS).

Aeroponic culture of W. somnifera

Chambers for aeroponic cultivation of plants measured 1.0 m×1.0 m×1.5 m (W×L×H) and were equipped with 6 nozzles powered by an external pump to spray nutrient solution every 4 min for a period of 1 min. A reservoir of 450 L of nutrient solution was maintained at the bottom of the chamber. The nutrient solution was prepared according to a general hydroponic recipe with a pH of 6.0. The aeroponic nutrient solution was made up by mixing solutions A and B prepared and mixed as follows: Solution A consisted of Ca(NO₃)₂.4H₂O (0.579 g/L), CaCl₂′6H₂O (0.278 g/L), 10% FeKH₂PO₄ (0.24 g/L), K₂SO₄ (0.193 g/L), MgSO₄.7H₂O (0.6 g/L), H₃BO₃ (0.003 g/L), 20% CuSO₄ (0.003 g/L), 20% MnSO₄H₂O (0.004 g/L), Na₂MoO₄.2H₂O (0.001 g/L), 20% ZnSO₄ 7H₂O (0.004 g/L). Solution A (900 mL) and Solution B (900 mL) were added to 140 L of water and mixed thoroughly and if necessary the pH of the solution adjusted to 5.6-6.0 with citric acid or KOH. Each box accommodated 20 plants. The mature plants were harvested and aerial parts (leaves and stems) and roots were collected separately. Roots were freeze-dried, while the aerial parts were air-dried.

Extraction and Isolation of 2,3-Dihydrowithaferin A-3β-O-Sulfate from W. somnifera

Dry powder (100 g) obtained from the aerial tissue of W. somnifera was extracted three times with MeOH (3×250 mL) at room temperature. After evaporation under reduced pressure, 19.8 g of the crude extract was obtained. A portion (1.98 g) of this extract was applied to a column of C-18 (30.0 g) and eluted successively with a gradient of 50-100% aqueous MeOH. The fraction eluted with 50% MeOH was further fractionated on a column of C-18 (30 g) with 40% aqueous MeOH as the eluent. Fractions were collected and combined based on their TLC profiles. Final purification was carried out on a column of silica gel and elution with CHCl₃-MeOH (8:2). Crystallization from MeOH yielded 2,3-dihydrowithaferin A-3β-O-sulfate (40.4 mg, 0.4%) as colorless crystals. Mp dec>167° C.; [α]²⁵_{D}+14.5 (c 0.21, MeOH); UV (MeOH) λₘₐₓ 214 nm; ¹H NMR (C₅D₅N, 500 MHz) δ 5.66 (1H, br. s, H-3), 4.84 (1H, d, J=12.0 Hz, H-27a), 4.74 (1H, d, J=12.0 Hz, H-27b), 4.43 (1H, br. s, H-4), 4.37 (1H, br. d, J=13.0 Hz, H-22), 3.62 (1H, br. dd, J=8.5, 16.0 Hz, H-2), 3.40 (1H, br. s, H-6), 3.25 (1H, d, J=16 Hz, H-2), 2.07 (3H, s, CH₃-28), 1.63 (3H, s, CH₃-19), 0.95 (3H, d, J=6.5 Hz, CH₃-21), 0.50 (3H, s, CH₃-18); ¹³C NMR (C₅D₅N, 500 MHz) δ 208.7 (qC, C-1), 166.4 (qC, C-26), 155.9 (qC, C-24), 127.3 (qC, C-25), 78.4 (CH, C-22), 75.5 (CH, C-4), 73.8 (CH, C-3), 65.0 (qC, C-5), 57.0 (Ch, C-6), 56.2 (CH₂, C-27), 56.0 (CH, C-14), 52.0 (CH, C-17), 49.7 (qC, C-10), 42.8 (qC, C-13), 42.5 (CH, C-9), 41.6 (CH₂, C-2), 39.2 (CH₂, C-16), 39.0 (CH, C-20), 31.3 (CH₂, C-23), 30.0 (CH₂, C-7), 29.9 (CH, C-8), 27.3 (CH₂, C-12), 24.5 (CH₂, C-15), 21.4 (CH₂, C-11), 20.1 (CH₃, C-28), 15.5 (CH₃, C-19), 13.6 (CH₃, C-21), 11.5 (CH₃, C-18); Negative HRESIMS m/z 567.2248 (calcd for C₂₈H₃₉O₁₀S, 567.2264).

### Cytotoxicity Assay

A standard tetrazolium dye [3-(4,5-di-methylthiazol-2-yl)-2,5-diphenyltetrazoliumbromide; MTT]-based colorimetric assay was used to measure the proliferation/survival of cells in triplicate wells using a 96-well plate-based format. Compounds 1 and 2 were formulated in DMSO and applied to cells such that final DMSO concentration did not exceed 0.2%. Cells were exposed continuously to test compounds for 24 or 72 h at which times related viable cell number per well was determined as previously described (Wijeratne, et al., J. Nat. Prod. (2003) 66: 1567-1573, incorporated herein by reference).

### Detection of Actin Aggregation

Cells were seeded in 8-well chamber slides at a density of 2×10⁴ cells per well and allowed to adhere for 48 h. Compounds 1 and 2 were freshly prepared as 5 mM stock solutions in DMSO and applied to cells at a final concentration of 4 μM in RPMI culture medium supplemented with 10% fetal bovine serum, Glutamax™, and penicillin/streptomycin. Control wells were treated with an equal volume of DMSO, not exceeding 0.2% in culture media. Cells were incubated for 4 or 24 h in the continuous presence of the indicated compounds, then washed twice with PBS, fixed with 4% paraformaldehyde/PBS (pH 7.6), and permabilized with 0.1% triton-X 100/PBS. Slides were blocked for 30 min at room temperature with 10% (v/v) goat serum and 1% bovine serum albumin (w/v) in PBS, then incubated with AlexaFluor 488-conjugated phalloidin to stain F-actin (Molecular Probes). To visualize nuclei, cells were counterstained with DAPI (1 μg/ml) in PBS for 3 min. After extensive washing, cells were visualized using an Olympus IX71 microscope with 100× objective and identical exposure conditions.

### Heat Shock Induction

Immortalized mouse embryo fibroblasts derived from homozygous Hsf1 knockout mice or their wild type littermates were exposed overnight to equitoxic concentrations of 1 or the known heat shock-inducing Hsp90 inhibitor gendanamycin. Whole cell lysates were prepared in non-ionic detergent buffer and immunoblotted for relative levels of Hsp72, a highly inducible member of the Hsp70 family of molecular chaperones, using monoclonal antibody C92F3A-5 (StressMarq Biosciences, Victoria, BC). Reactivity was detected using peroxidase-conjugated secondary antibody and chemiluminescent detection. To evaluate the relative ability of compounds to induce a heat shock response at the transcriptional level, a reporter cell line was used as previously described (Turbyville, et al., J. Nat. Prod. (2006) 69: 178-184, incorporated herein by reference). These cells are stably transduced with a plasmid encoding enhanced green fluorescent protein (EGFP) under the control of a minimal heat shock response element derived from the promoter region of the Hsp70B gene. They demonstrate a robust, concentration-dependent fluororescent response to known heat shock-modulating drugs such as Hsp90 inhibitors and can be used as a sensitive and specific system to non-destructively monitor induction of the heat shock response in live cells.

### Conversion of 2,3-Dihydrowithaferin A-3β-19-Sulfate to Withaferin a in Cell Culture Media

Stock solutions of 2,3-dihydrowithaferin A-3β-O-sulfate in DMSO (50 μL) were diluted into cell culture medium (950 μL) to achieve the indicated starting concentration, mixed thoroughly and the solution incubated in a CO₂ incubator at 37° C. Aliquots (100 μL) were withdrawn at 4, 16, and 24 h and subjected to HPLC analysis for 2,3-dihydrowithaferin A-3β-O-sulfate (RR_{T}=18.5 min) and withaferin A (RR_{T}=23.5 min) on a Kromasil C₁₈ RP column (250×4.6 mm, 5 mm) with gradient elution using 40-100% aqueous MeOH and using an ELSD detector. An external standard curve method was used to calculate the concentration of each compound in the sampled aliquots (Khajuria, et al., J. Sep. Sci. (2004) 27: 541-546, incorporated herein by reference).

Synthesis of Epi-Withaferin A from Withaferin A

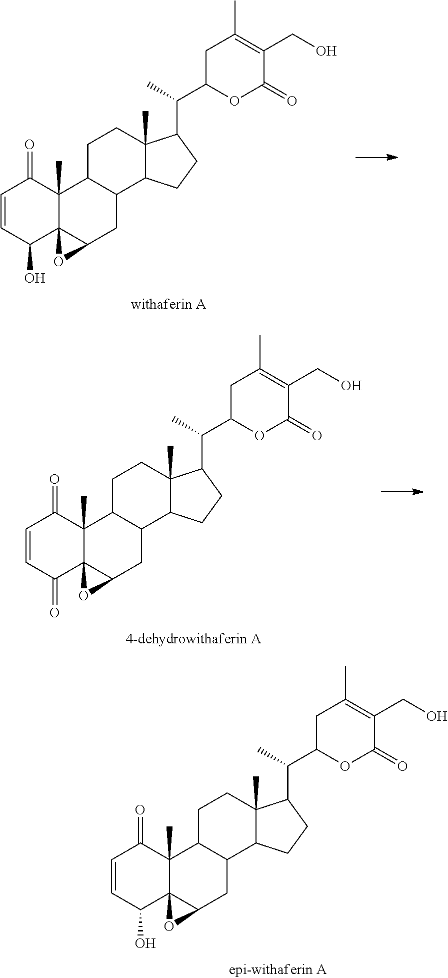

4-dehydrowithaferin A was prepared by manganese dioxide oxidation of withaferin A as described in the literature (Lavie, et al., J. Chem. Soc. (1965) 7517-7531). Briefly, to a solution of withaferin A (30 mg) in chloroform/ethyl acetate (5:7, 2.0 mL) was added freshly prepared manganese dioxide (MnO₂, 300 mg) and stirred at 25° C. After 16 hours, the reaction mixture was filtered, the filtrate was evaporated under reduced pressure, and the residue was separated via preparative thin layer chromatography (silica gel) using 8% methanol in dichloromethane as eluant to give 4-dehydrowithaferin A (18.4 mg, 62% yield).

To a stirred solution of 4-dehydrowithaferin A (6.0 mg) in methanol (1.0 mL) and tetrahydrofuran (0.5 mL) was added CeCl₃.7H₂O (130 mg). The reaction mixture was then kept in an ice bath and NaBH₄ (ca. 0.5 mg) was added and stirred at 0° C. After 30 minutes, a small ice cube was added to the reaction mixture. Solvents were evaporated under reduced pressure, and the residue was separated via preparative thin layer chromatography (silica gel) using 6% methanol in dichloromethane as eluant to give epi-withaferin A (4.2 mg, 70% yield) as a white solid; mp 227-228° C.; [α]²⁵_{D}+29.9 (c 1.0, CHCl₃); NMR (500 MHz, CDCl₃) δ: 6.80 (dd, J=10.1, 1.5 Hz, 1H, H-3), 5.97 (dd, J=10.1, 2.5 Hz, 1H, H-2), 4.64 (brs, 1H, H-4), 4.37 (dt, J=13.5, 3.3 Hz, 1H, H-22), 4.32 (d, J=12.5 Hz, 1H, H-27a), 4.27 (d, J=12.5 Hz, 1H, H-27b), 3.65 (brs, 1H, H-6), 2.45 (dd, J=13.6, 7.2 Hz, 1H, H-23a), 2.10 (brd, 1H, H-7a), 2.00 (s, 3H, H₃-28), 1.96-1.89 (m, 4H), 1.78 (brs, 1H), 1.67-1.58 m, 2H), 1.49-1.42 (m, 2H), 1.31 (m, 1H), 1.18 (s, 3H, H₃-18), 1.15-1.00 (m, 4H), 0.94 (d, J=6.6 Hz, 3H, H₃-21), 0.88 (m, 1H), 0.66 (s, 3H, H₃-19); ¹³C NMR (125 MHz, CDCl₃) δ: 201.4, 167.1, 153.3, 148.2, 128.4, 125.6, 78.7, 65.7, 64.3, 57.0, 55.9, 55.3, 51.9, 47.5, 45.5, 42.5, 39.3, 38.7, 30.6, 29.8, 29.7, 27.2, 24.1, 22.2, 19.9, 13.8, 13.2, 11.6; HRFABMS m/z 471.2764 [M+H]⁺ (calcd for C₂₈H₃₉O₆ 471.2747).

Synthesis of 4,27-Di-O-Acetyl Epi-Withaferin A from Epi-Withaferin A

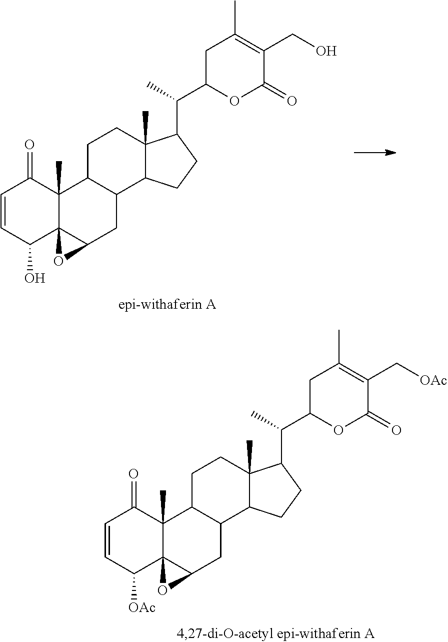

To a solution of epi-withaferin A (1.0 mg) in pyridine (0.1 mL) was added acetic anhydride (0.1 mL) and stirred at 25° C. After 14 hours, ethanol (15 mL) was added to the reaction mixture. The volatiles were evaporated under reduced pressure, and the residue was separated via preparative thin layer chromatography (silica gel) using 6% methanol in dichloromethane as eluant to give 4,27-di-O-acetyl epi-withaferin A (1.1 mg, 93% yield); mp 214-216° C.; [α]²⁵_{D}+36.8 (c 1.1, CHCl₃); ¹HNMR (600 MHz, CDCl₃) δ: 6.66 (dd, J=10.1, 1.5 Hz, 1H, H-3), 6.05 (dd, J=10.1, 2.4 Hz, 1H, H-2), 5.87 (brs, 1H, H-4), 4.88 (d, J=11.8 Hz, 1H, H-27a), 4.84 (d, J=11.8 Hz, 1H, H-27b), 4.38 (dt, J=13.1, 3.3 Hz, 1H, H-22), 3.53 (brs, 1H, H-6), 2.50 (dd, J=17.6, 13.3 Hz, 1H, H-23a), 2.09 (s, 3H, OAc), 2.05 (s, 3H, H₃-28), 2.03 (s, 3H, OAc), 2.01-1.92 (m, 4H), 1.67-1.33 (m, 6H), 1.28 (s, 3H, H₃-18), 1.23-1.01 (m, 4H), 0.98 (d, J=6.6 Hz, 3H, H₃-21), 0.94-0.81 (m, 2H), 0.70 (s, 3H, H₃-19); APCI-MS (+) m/z 555 [M+1]⁺.

### Synthesis of 27-O-Acetyl Epi-Withaferin A

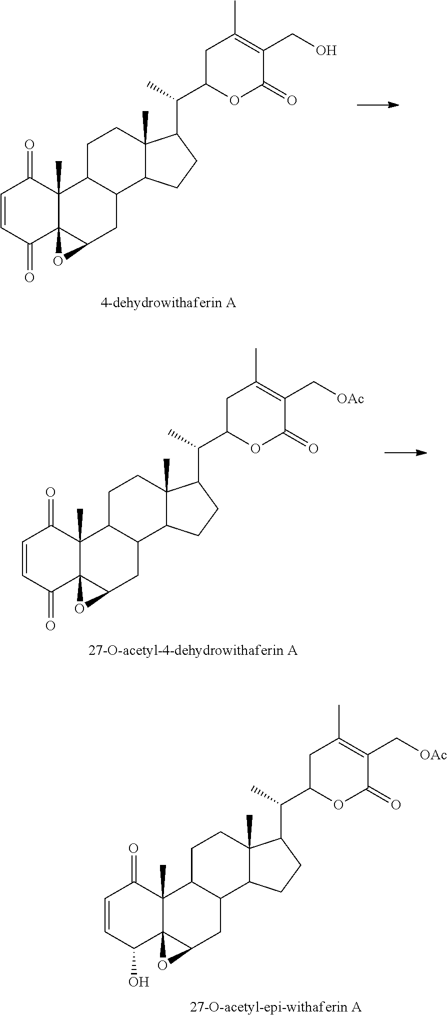

To a stirred solution of 4-dehydrowithaferin A (5 mg) in pyridine (0.2 mL) was added acetic anhydride (0.1 mL), and the reaction was stirred at 25° C. for 18 hours. Pyridine and excess acetic anhydride were evaporated under reduced pressure and azeotroped with ethanol. The resulting residue was then purified via preparative thin layer chromatography using 4% methanol in dichloromethane as eluant to give 27-O-acetyl-4-dehydrowithaferin A (5.25 mg, 96% yield). A portion of 27-O-acetyl-4-dehydrowithaferin A (3.0 mg) was then dissolved in a mixture of tetrahydrofuran (0.2 mL) and methanol (0.2 mL). CeCl₃.7H₂O (65 mg) was added, and the mixture was stirred at 0° C. for 5 minutes. To this solution NaBH₄ (ca 0.5 mg) was added, and the mixture was stirred at 0° C. for 10 minutes further. A small ice cube was added to the reaction mixture, solvents were evaporated under reduced pressure, and the residue was partitioned between water and ethyl acetate. The ethyl acetate layer was dried over anhydrous Na₂SO₄, evaporated under reduced pressure, and the residue was separated via preparative thin layer chromatography (silica gel) using 2% methanol in dichloromethane as eluant to give 27-O-acetyl epi-withaferin A (2.5 mg, 70% yield) as a white solid, mp 188-190° C.; ¹H NMR (500 MHz, CDCl₃) δ: 6.83 (dd, J=10.2, 1.4 Hz, 1H, H-3), 6.00 (d, J=10.2, 2.5 Hz, 1H, H-2), 4.88 (d, J=11.9 Hz, 1H, H-27a), 4.85 (d, J=11.9 Hz, 1H, H-27b), 4.71 (s, 1H, H-4), 4.38 (dt, J=13.2, 3.3 Hz, 1H, H-22), 3.63 (s, 1H, H-6), 2.50 (dd, J=17.6, 14.5 Hz, 1H, H-23a), 2.12 (m, 1H, H-7a), 2.05 (s, 3H, H₃-28), 2.03 (s, 3H, OAc), 1.99 (dd, J=13.2, 3.3 Hz, 1H), 1.93 (brd, J=9.9 Hz, 1H), 1.68-1.45 (m, 4H), 1.34 (m, 1H), 1.28-1.1.22 (m, 3H), 1.21 (s, 3H, H₃-18), 1.18-1.03 (m, 4H), 0.98 (d, J=6.7 Hz, 3H, H₃-21), 0.94-0.81 (m, 2H), 0.69 (s, 3H, H₃-19); APCI-MS (+) m/z 513 [M+1]⁺.

### Synthesis of 4-O-Acetyl Epi-Withaferin A from Withaferin A

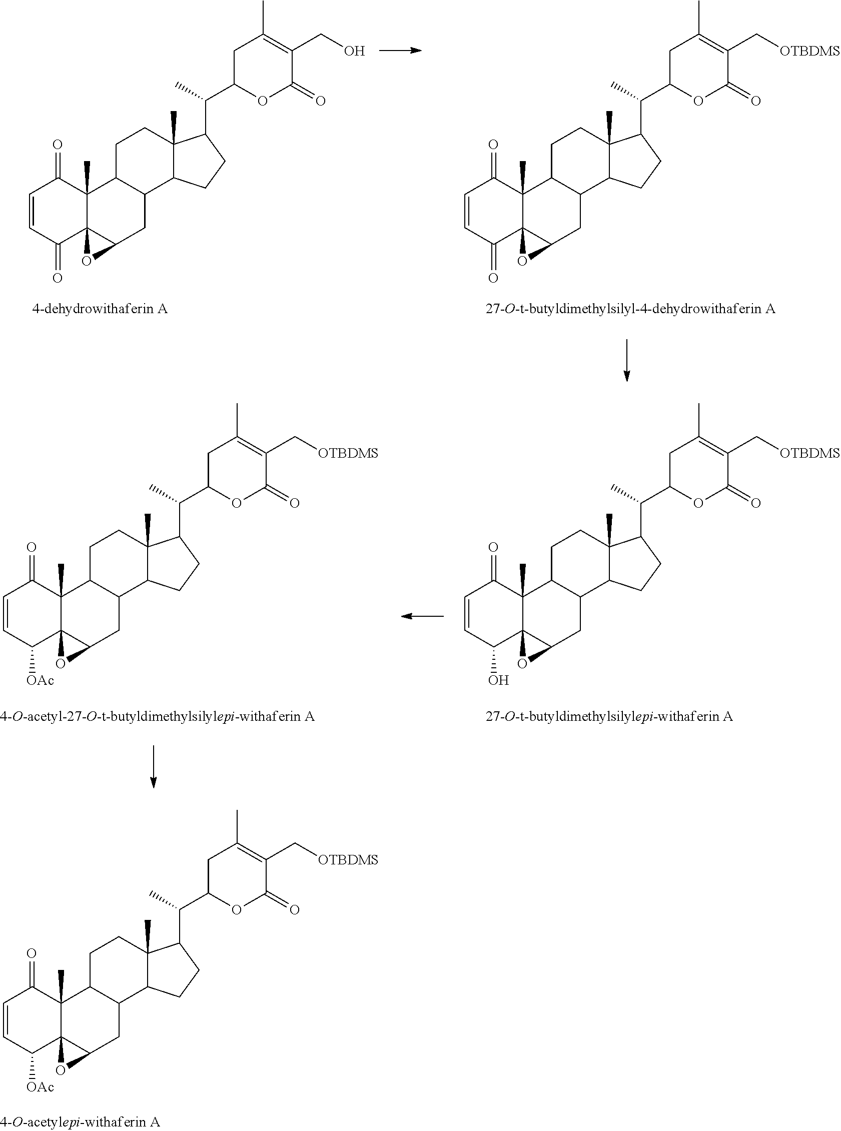

To a solution of 4-dehydrowithaferin A (11.3 mg) in DMF (0.5 mg) were added t-butyldimethylsilyl chloride (36.4 mg) and 4-pyrrolidinopyridine (42.9 mg) and stirred under atmosphere of nitrogen for 1 hour at 60° C. The reaction mixture was then diluted with ethyl acetate and washed with brine. The ethyl acetate solution was evaporated under reduced pressure, and the residue was separated via preparative thin layer chromatography using dichloromethane as eluant to give 27-O-t-butyldimethylsilyl-4-dehydrowithaferin A (9.5 mg, 68% yield). This compound was then dissolved in tetrahydrofuran (0.2 mL) and methanol (0.2 mL). CeCl₃.7H₂O (125 mg) was added, and the reaction was stirred at 0° C. for 5 minutes. To this solution was added NaBH₄ (ca 1.0 mg), and the reaction was stirred at 0° C. After 10 minutes, a small ice cube was added to the reaction mixture, solvents were evaporated under reduced pressure, and the residue was partitioned between water and ethyl acetate. The ethyl acetate layer was dried over anhydrous Na₂SO₄, evaporated under reduced pressure, and the residue was separated via preparative thin layer chromatography (silica gel) using 2% methanol in dichloromethane as eluant to give 27-O-t-butyldimethylsilyl epi-withaferin A (7.5 mg, 70% yield) as a white solid, APCI-MS (+) m/z 585 [M+1]⁺. 27-O-t-Butyldimethylsilyl epi-withaferin A was then acetylated using acetic anhydride and pyridine to give 4-O-acetyl-27-O-t-butyldimethylsilyl epi-withaferin A (8.0 mg, 99.5% yield) as a white solid (APC)-MS (+) m/z 627 [M+1]⁺). 4-O-acetyl-27-O-t-butyldimethylsilyl epi-withaferin A (8.0 mg) was then dissolved in tetrahydrofuran (0.5 mL) and methanol (0.3 mL) and kept in an ice bath. To this solution was added 2 N HCl (0.15 mL), and the reaction was stirred at 0° C. After 1 hour, the reaction mixture was diluted with water. Methanol and tetrahydrofuran were evaporated under reduced pressure, and the water remaining was extracted with ethyl acetate (3×15 mL). The combined ethyl acetate extracts were washed with water, dried over anhydrous Na₂SO₄, evaporated under reduced pressure, and the residue was separated via preparative thin layer chromatography (silica gel) using 5% methanol in dichloromethane as eluant to give 4-O-acetyl epi-withaferin A as a white solid (5.3 mg, 70% yield), mp 236-38° C.; NMR (600 MHz, CDCl₃) δ: 6.66 (dd, J=10.4, 1.5 Hz, 1H, H-3), 6.05 (dd, J=10.4, 2.4 Hz, 1H, H-2), 5.87 (brs, 1H, H-4), 4.39 (brd, J=13.4, 3.3 Hz, 1H, H-22), 4.37 (d, J=12.5 Hz, 1H, H-27a), 4.32 (d, J=12.5 Hz, 1H, H-27b), 3.53 (brs, 1H, H-6), 2.48 (dd, J=16.2, 13.9 Hz, 1H, H-23a), 2.11 (brd, 1H, H-7a), 2.09 (s, 3H, OCH₃), 2.01 (s, 3H, H₃-28), 1.97-1.93 (m, 4H), 1.54-1.45 (m, 2H), 1.34 (m, 1H), 1.28 (s, 3H, H₃-18), 1.23-1.00 (m, 6H), 0.98 (d, J=6.6 Hz, 3H, H₃-21), 0.94-0.84 (m, 2H), 0.69 (s, 3H, H₃-19); APCI-MS (+) m/z 513 [M+1]⁺.

Synthesis of 27-O-Acetylwithaferin A from Withaferin A

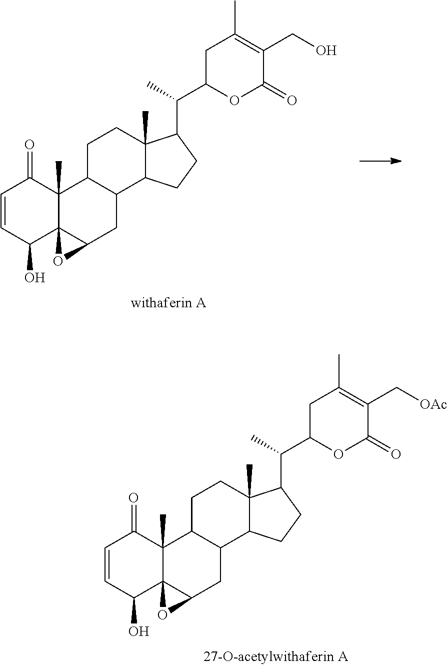

To a solution of withaferin A (10.0 mg) in pyridine (0.1 mL) was added acetic anhydride (2.4 μL), and the reaction was stirred at 25° C. After 2 h, ethanol (15 mL) was added to the reaction mixture. The volatiles were evaporated under reduced pressure, and the residue was separated via preparative thin layer chromatography (silica gel) using 6% methanol in dichloromethane as eluant to give 27-O-acetylwithaferin A (8.5 mg, 72% yield) as a white solid; mp 218-220° C.; ¹H NMR (500 MHz, CDCl₃) δ: 6.90 (dd, J=9.9, 5.8 Hz, 1H, H-3), 6.18 (d, J=9.9 Hz, 1H, H-2), 4.88 (d, J=11.8 Hz, 1H, H-27a), 4.84 (d, J=11.8 Hz, 1H, H-27b), 4.38 (dt, J=13.6, 3.3 Hz, 1H, H-2), 3.74 (dd, J=5.8, 2.1 Hz, 1H, H-6), 3.22 (s, 1H, H-4), 2.51 (dd, J=13.2, 10.9 Hz, 2H), 2.12 (ddd, J=14.9, 6.3, 2.6, 1H, H-7a), 2.05 (s, 3H, H₃-28), 2.04 (s, 3H, OAc), 1.96 (m, 2H), 1.93 (dt, J=9.6, 3.3 Hz, 1H), 1.82 (dt, J=14.2, 3.6 Hz, 1H), 1.69-1.59 (m, 2H), 1.53-1.43 (m, 2H), 1.39 (s, 3H, H₃-18), 1.25 (m, 3H), 1.18-1.01 (m, 2H), 0.98 (d, J=6.6 Hz, 3H, H₃-21), 0.91-0.82 (m, 2H), 0.69 (s, 3H, H₃-19); APCI-MS (+) m/z 513 [M+1]⁺.

### Assessment of Withanolides in Neuroprotection Models

As described above, withaferin A showed neuroprotective effects in certain cell-based assays. In further experiments, additional withanolides are assessed in one or more of these cell-based assays.

In other experiments, neuroprotective effects of withanolide(s) are assessed in one or more in vivo animal models, e.g., in rodents such as mice or rats. One such model is an ischemic stroke model, e.g., a model involving occlusion of the middle cerebral artery (MCAO), optionally followed by reperfusion, e.g., as described by Arboleda-Velasquez, J F, et al. (Arboleda, J. F., et al., Proc. Natl. Acad. Sci. 105(12):4856-4861, 2008; Huang, Z., et al., Science, 265: 1883-1885, 1994; Huang, Z., et al., J. Cereb. Blood Flow Metab. 17: 1143-1151, 1997.). Another model is a spinal cord injury model. See, e.g., Basso, D M, et al., A sensitive and reliable locomotor rating scale for open field testing in rats. J. Neurotrauma, 12(1):1-21, 1995; Basso, D M., et al., Graded histological and locomotor outcomes after spinal cord contusion using the NYU weight-drop device versus transection. Exp. Neurol., 139(2): 244-256, 1996.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A compound of formula: 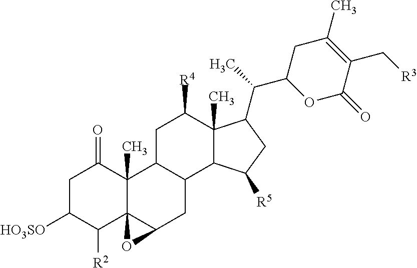
or a pharmaceutically acceptable salt thereof; wherein
R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R³, R⁴ and R⁵ are each independently hydrogen or —OR^{S}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

2. The compound of claim 1 wherein R² is hydrogen, —OH, —OR^{B}, —OSO₃H, or —OAc.

3-6. (canceled)

7. The compound of claim 1 wherein R³ is hydrogen, —OH, —OR^{C}, —OSO₃H, or —OAc.

8-11. (canceled)

12. The compound of claim 1 wherein R⁴ is hydrogen, —OH, —OR^{C}, —OSO₃H, or —OAc.

13-16. (canceled)

17. The compound of claim 1 wherein R⁵ is hydrogen.

18-21. (canceled)

22. The compound of claim 1 wherein R⁴ and R⁵ are both hydrogen.

23. The compound of claim 1 of formula:

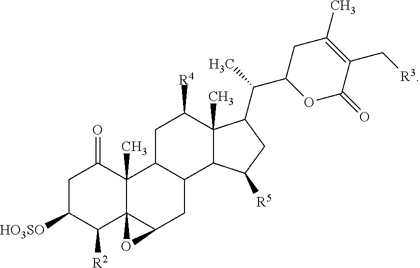

24. The compound of claim 1 of formula:

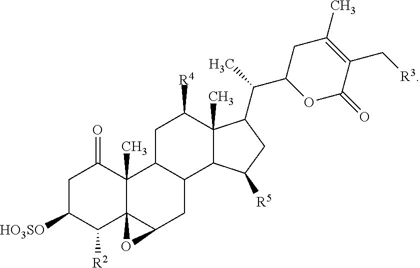

25. The compound of claim 1 of formula:

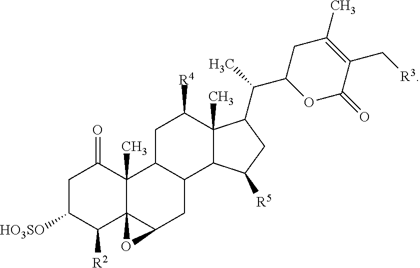

26. The compound of claim 1 of formula:

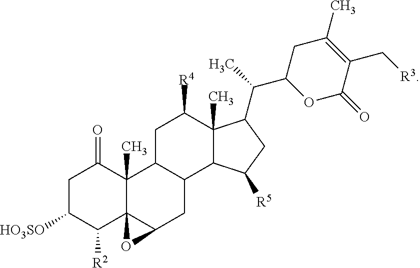

27. The compound of claim 1 of formula:

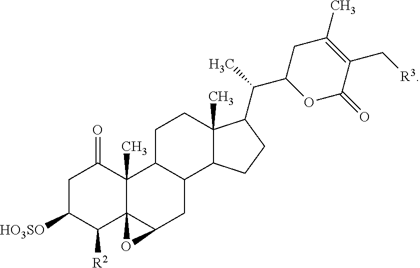

28-29. (canceled)

30. The compound of claim 1 with one of the following structures:

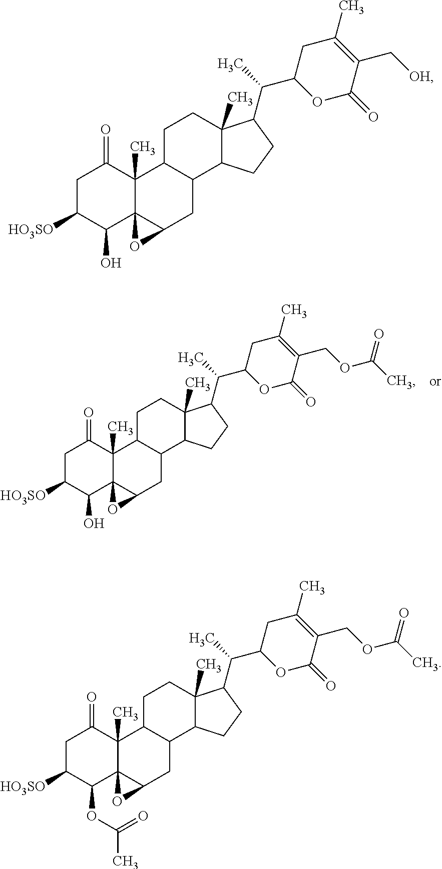

31. (canceled)

32. A compound of formula: 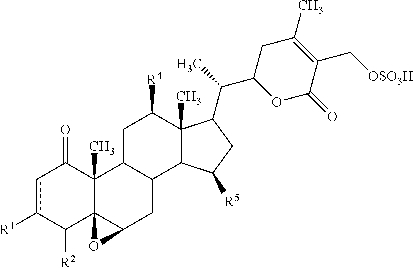
or a pharmaceutically acceptable salt thereof; wherein [symbol]
=denotes a single or double bond;
R¹ is hydrogen or —OR^{A}, where R^{A} is hydrogen, —SO₃H; —PO₃H₂; —C(═O)R^{D}; —C(═O)N(R^{D})₂; —CO₂R^{D}; —SOR^{A}; —SO₂R^{D}; —C(R^{D})₃; wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
R² is —OR^{B}, where R^{B} is hydrogen, —SO₃H; —PO₃H₂; —C(═O)R^{D}; —C(═O)N(R^{D})₂; —CO₂R^{D}; —SOR^{D}; —SO₂R^{D}; or —C(R^{D})₃;
R⁴ and R⁵ are each independently hydrogen or —OR^{S}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃.

33. The compound of claim 32 wherein [symbol] is a double bond.

34-56. (canceled)

57. The compound of claim 32 of formula:

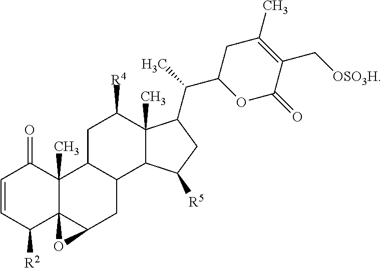

58-78. (canceled)

79. The compound of claim 32 with one of the following structures:

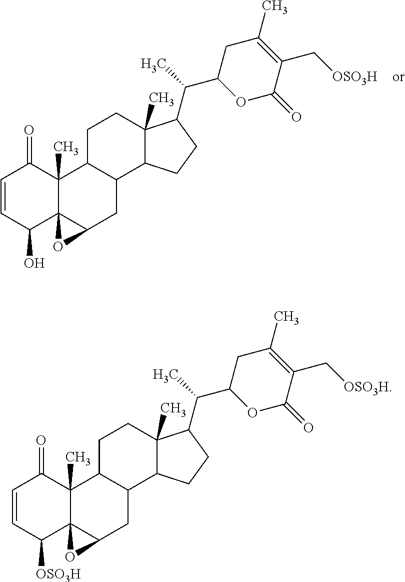

80. A compound of formula: 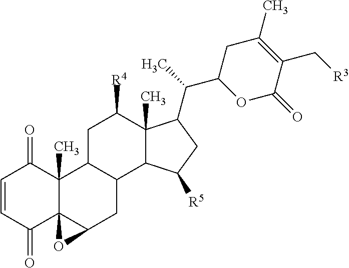
or a pharmaceutically acceptable salt thereof; wherein
R³, R⁴ and R⁵ are each independently hydrogen or —OR^{S}, where each occurrence of R^{C} is independently hydrogen, —SO₃H; —PO₃H₂; —C(═O)R^{D}; —C(═O)N(R^{D})₂; —CO₂R^{D}; —SOR_{A}; —SO₂R_{C}; or —C(R^{D})₃, wherein each occurrence of R^{D} is independently a hydrogen, a halogen, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

81-96. (canceled)

97. The compound of claim 80 with one of the following structures:

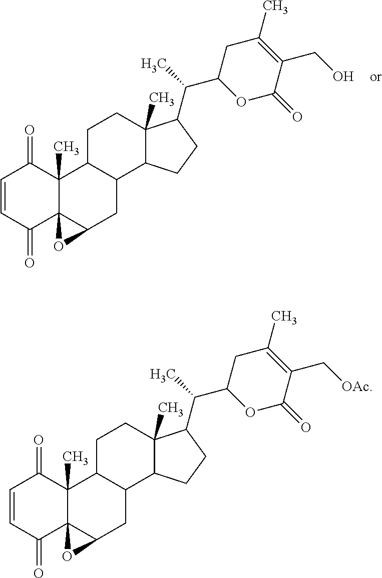

98-137. (canceled)

138. A pharmaceutical composition comprising a compound of claim 1; and a pharmaceutically acceptable excipient or vehicle.

139. (canceled)

140. A method of treating a subject having a proliferative disease, the method comprising administering a compound of claim 1 to a subject.

141-142. (canceled)

143. A method of treating a subject having cancer, the method comprising administering a compound of claim 1 to a subject, wherein said compound is a radiosensitizer.

144-147. (canceled)

148. A method of treating a subject having a neurodegenerative disease, the method comprising administering a compound of claim 1 to a subject.

149. (canceled)

150. A method of treating a subject having an inflammatory disease, the method comprising administering a compound of claim 1 to a subject.

151-153. (canceled)

154. A method of treating a subject having a protein aggregation disorder, the method comprising administering a compound of claim 1 to a subject.

155-156. (canceled)

157. A method of activating a heat shock response in a cell, the method comprising contacting a cell with an amount of a compound of claim 1 sufficient to induce a heat shock response.

158-159. (canceled)

160. A method of isolating a withanolide, the method comprising steps of:
growing Withania somnifera aeroponically;
extracting aeroponically-grown W. somnifera with a solvent to provide an extract; and
isolating said withanolide from said extract.

161-182. (canceled)
